(19) United States
(12) Patent Application Publication
(10) Pub. No.: US 2026/0334463 A1
(43) Pub. Date: Oct. 29, 2026

(54) SYSTEM FOR FREEZING AND/OR VITRIFYING BIOLOGICAL SPECIMEN

(71) Applicant: Insphero AG, Schlieren (CH)

(72) Inventors: Daniel Felder, Jona (CH); Lukas Müller, Hittnau (CH); Domenico Leonardo, Thalwil (CH); Rafael Herzog, Uznach SG (CH); Manuel Altmeyer, Jona (CH); Simon Hutter, Schlieren (CH); Wolfgang Moritz, Schlieren (CH)

(21) Appl. No.: 19/124,262

(22) PCT Filed: Oct. 26, 2023
(86) PCT No.: PCT/EP2023/079952

§ 371 Date: Apr. 24, 2025

(30) Foreign Application Priority Data
Oct. 26, 2022 (EP) 22203843.2
Feb. 7, 2023 (EP) 23155412.2

(51) Int. Cl. A01N 1/145

(52) U.S. Cl. CPC A01N 1/145

(57) ABSTRACT

The present invention relates to a system for freezing and/or vitrifying biological specimen. The system comprises an encasement (40) arranged to accommodate a cryogenic liquid (C), a support (4) for a recipient vessel, and optionally an upper reservoir (2) with a first overflow rim (5) that allows cryogenic liquid to flow from the upper reservoir into a lower reservoir (12) with a second overflow rim (6). The support may comprise one or more cavities (10) that are suitable to accommodate wells or vials of the recipient vessel. The support may comprise a material that has a wicking effect on the cryogenic liquid, or be disposed on spacers. The system may further comprise pipetting means. The disclosure further relates to a recipient vessel, a vent plate for thawing biological specimen, as well as methods for freezing and/or vitrifying, for storing and for thawing biological specimen.

### FIELD OF THE INVENTION

The present application relates to systems and methods for freezing and/or vitrifying biological specimen.

### BACKGROUND

Biological specimen play an important role in biological sciences and applications. Being living material, they have a limited lifespan, and do therefore oftentimes require proper conservation.

Snap freezing is the process of rapid cooling of a substance for the purpose of preservation. It is widely used in the scientific industries, when a sample is very quickly lowered to ultralow temperatures, e.g., below −70 °C. This is usually accomplished by submerging the sample in liquid nitrogen, or in a mixture of dry ice and ethanol. This process is particularly useful to freeze biomolecules, like purified proteins so as to prevent ice crystals from forming and damaging the protein.

For other biological specimen like cells or microtissues, slow-freezing is oftentimes used, where the specimen, contained in an aqueous medium, are cooled to below freezing point. At some stage, ice masses containing pure crystalline water will form. The biological material and all solutes are thus confined and the concentrations of e.g. sugars, salts and/or cryoprotectants increase, while the volume of the unfrozen fraction decreases. The increase in osmotic strength causes an efflux of water from the cells. Slow cooling is needed in order to allow sufficient efflux of water to minimize the chance of intracellular ice formation. As cooling continues, the viscosity of the unfrozen fraction ultimately becomes too high for any further crystallization. The remaining unfrozen fraction turns into an amorphous solid that contains no ice crystals.

It would be desirable to subject biological specimen like cells or microtissues also to snap freezing in order to avoid the above problems. However, due to the high thermal capacity of receptacles in which biological specimens are usually stored or transported, the high freezing and warming rates required for snap freezing and thawing can not be achieved.

It is hence one object of the present invention to provide improved methods and systems for fast freezing and/or vitrification of biological specimen.

These and other objects are solved by the features of the independent claims. The dependent claims disclose embodiments of the invention which may be preferred under particular circumstances. Likewise, the specification discloses further embodiments of the invention which may be preferred under particular circumstances.

### BRIEF DESCRIPTION OF THE FIGURES

FIG. 1A shows a lateral cross section of a system for freezing and/or vitrifying biological specimen according to the invention in a schematized and simplified form. In the context of this specification, this system as well as others described herein will also be called “cryobox” or “cryochamber”. The system comprises an encasement 40 arranged to accommodate a cryogenic liquid C. The encasement 40 comprises, as an optional feature, a cover lid 14 with an opening 15 for filling in cryogenic liquid, and contains a support 4 for a recipient vessel 16 suitable and/or arranged for accommodating one or more biological specimen.

FIG. 1B shows a lateral cross section of another system for freezing and/or vitrifying biological specimen according to the invention in a schematized and simplified form. The system comprises an encasement 40 arranged to accommodate a cryogenic liquid C. The encasement 40 comprises, as an optional feature, a cover lid 14 with an opening 15 for filling in cryogenic liquid, and contains a support 4 for a recipient vessel 16 suitable and/or arranged for accommodating one or more biological specimen.

The support 4 for a recipient vessel 16 comprises a material that has an open-cell sponge-like structure and maintains elastic deformability at low temperatures (e.g., −70 °C or lower). In such way, it exerts a wicking effect on the cryogenic liquid. Accordingly, cryogenic liquid C comprised in the encasement 40 and/or in the lower reservoir 12 is soaked into the support 4, and the recipient vessel and its contents disposed thereon are cooled from sideways and from underneath.

The system further comprises, as an optional feature, a positioning grid 11 which allows secure and reproducible positioning of the support 4 for a recipient vessel.

As an optional feature, the system comprises a reservoir 12 with an overflow rim 6 which creates an overflow compartment 7. The overflow rim 6 is arranged to allow the cryogenic liquid to flow from the reservoir 12 into the overflow compartment 7 if the level height of the cryogenic liquid in the reservoir 12 exceeds the height of the overflow rim 6. For that purpose, the height of the overflow rim 6 aligns with the height of the support 4 for the recipient vessel.

The system further comprises, as an optional feature, a compartment 8 arranged below the reservoir 12. The compartment 8 is in fluid communication with the overflow compartment 7 and with the support 4, and is suitable and/or arranged to accommodate a cryogenic liquid.

The compartment 8 may comprise a material that has an open-cell sponge-like structure and maintains elastic deformability at low temperatures. In such way, it exerts a wicking effect on the cryogenic liquid. Accordingly, cryogenic liquid C in the overflow compartment is soaked into the compartment 8 and into the support 4, and the recipient vessel and its contents disposed thereon are cooled from sideways and from underneath.

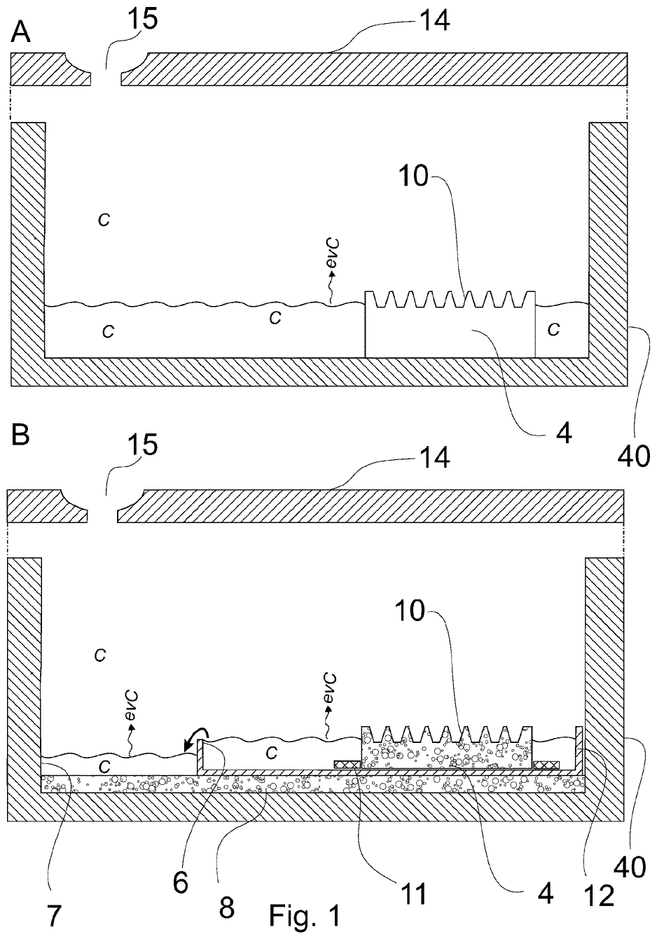

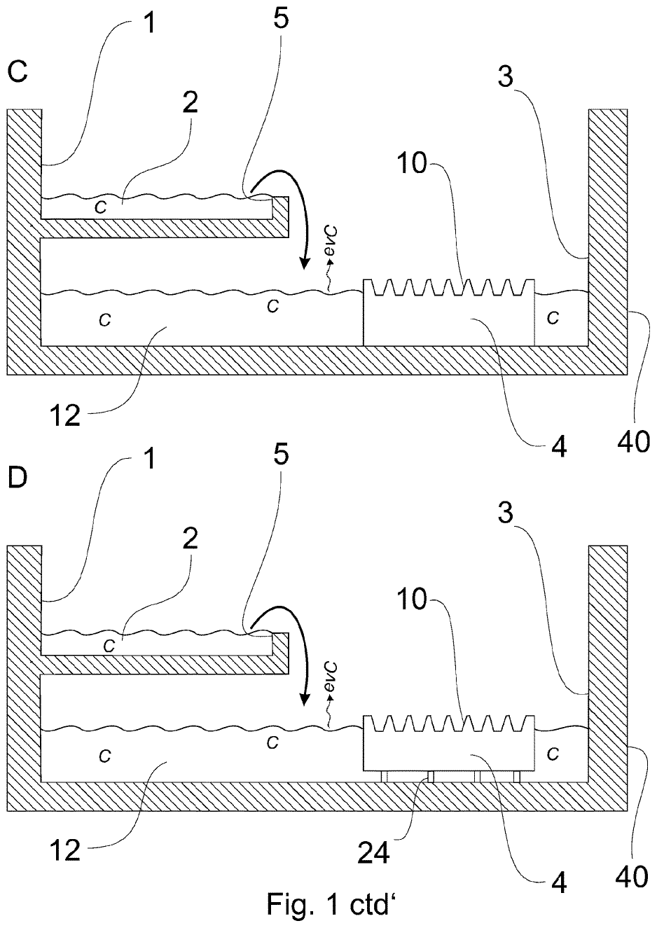

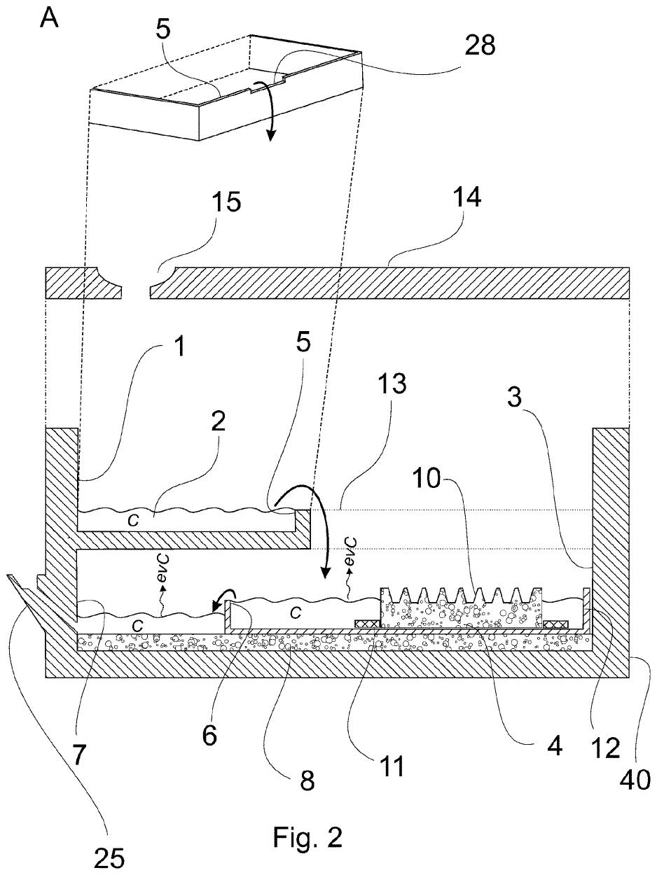

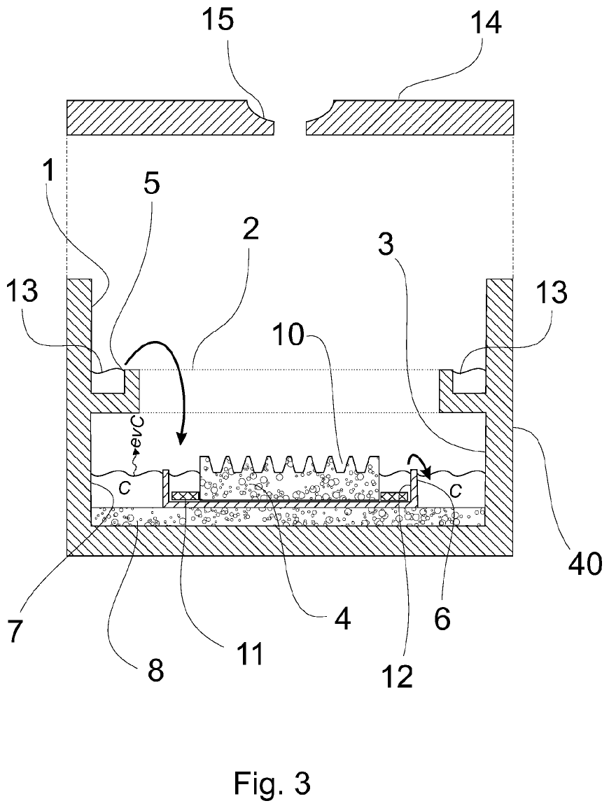

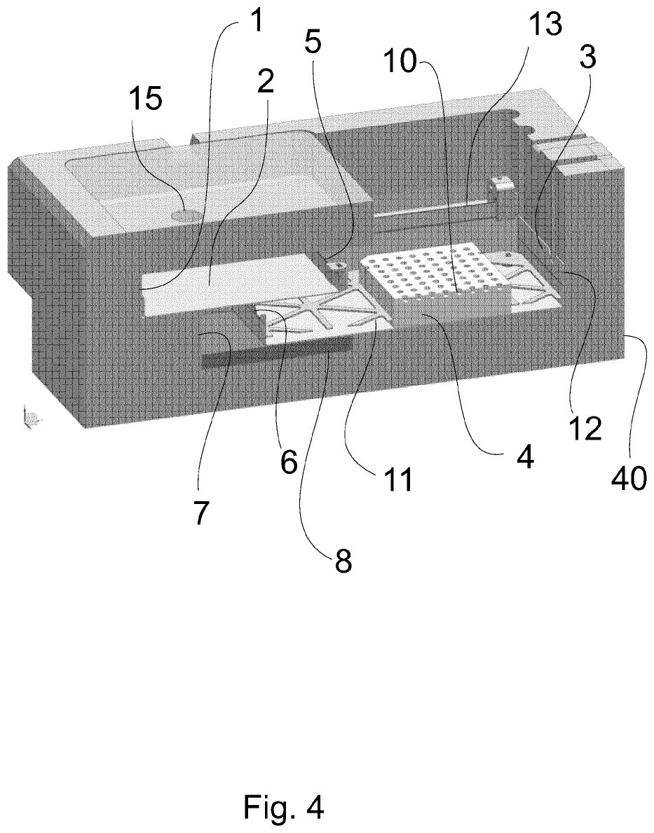

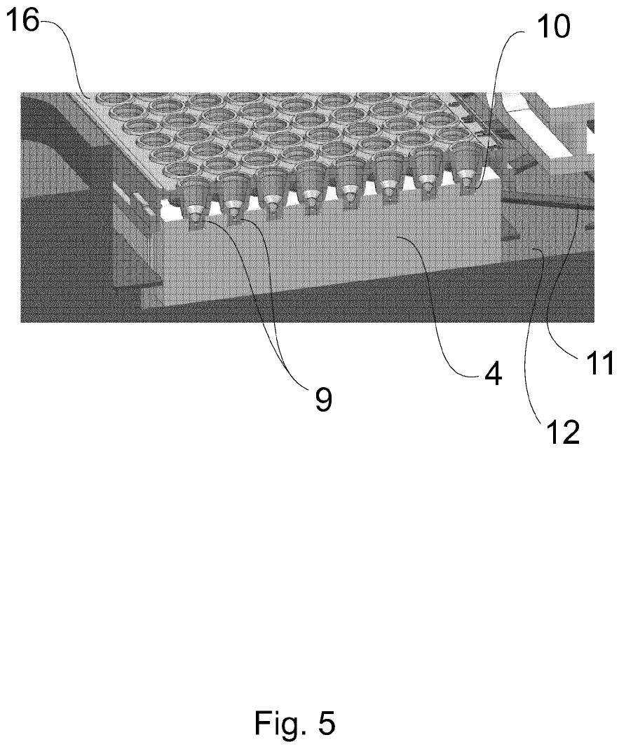

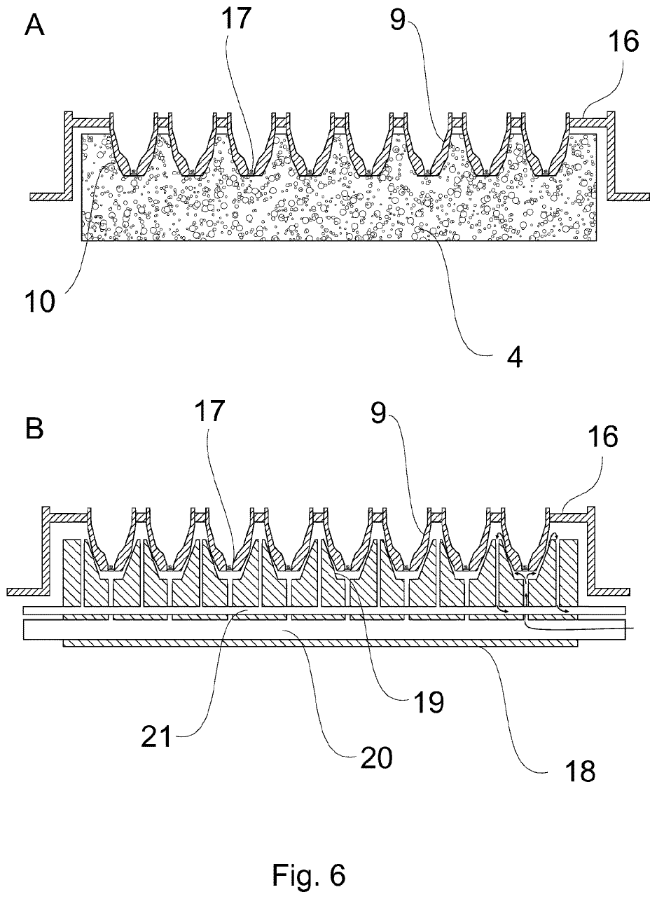

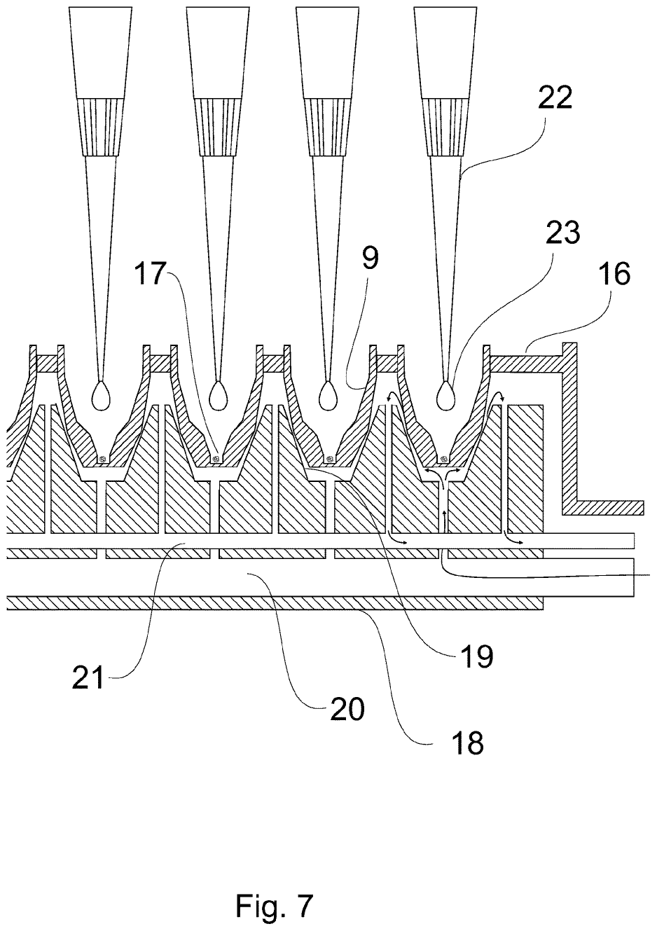

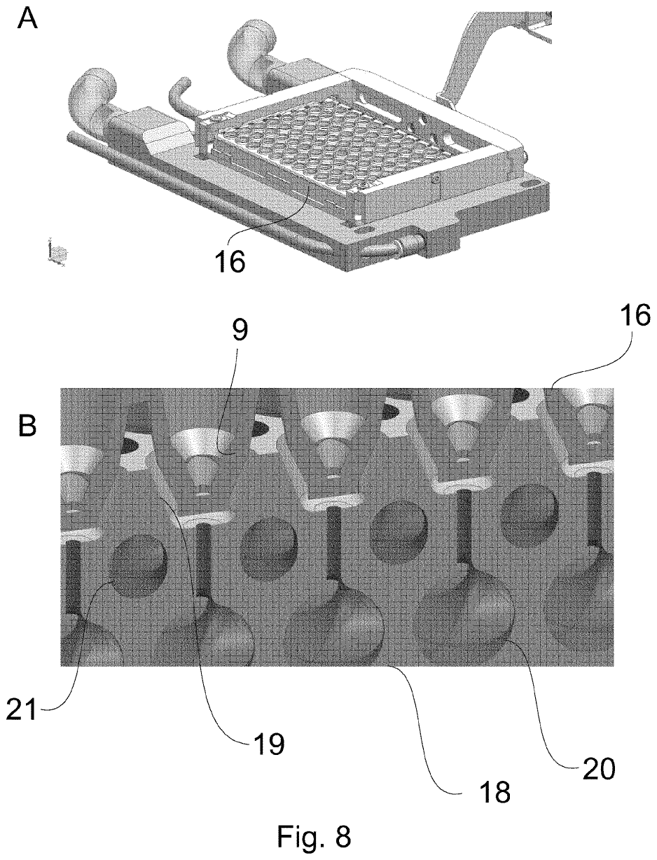

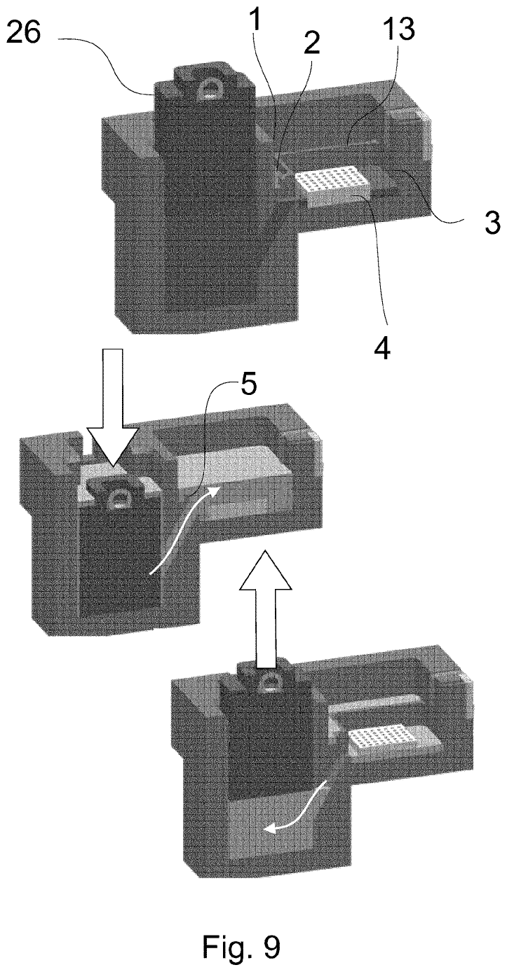

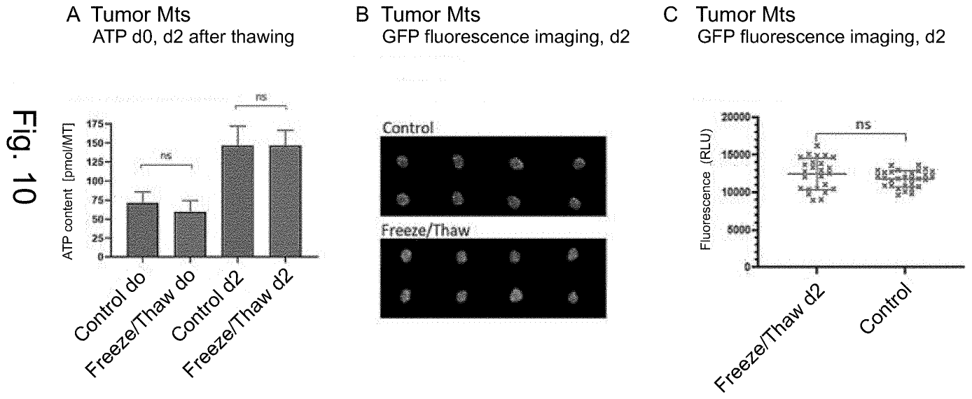

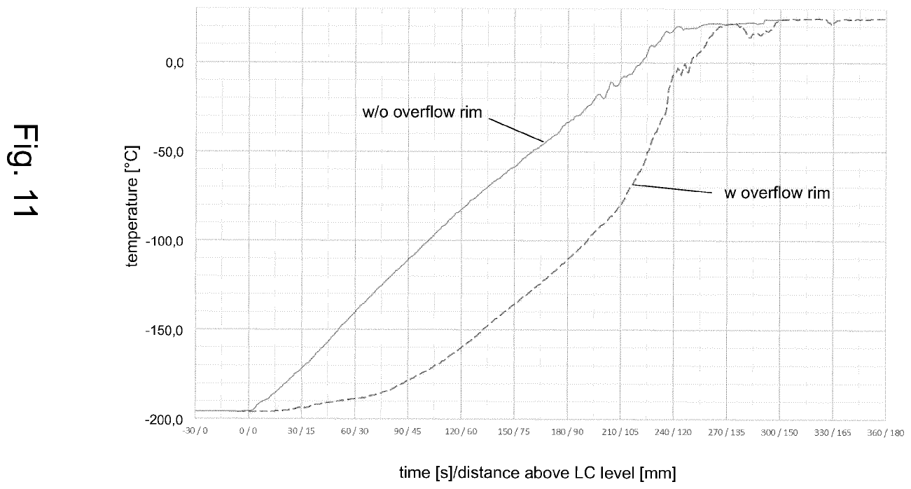

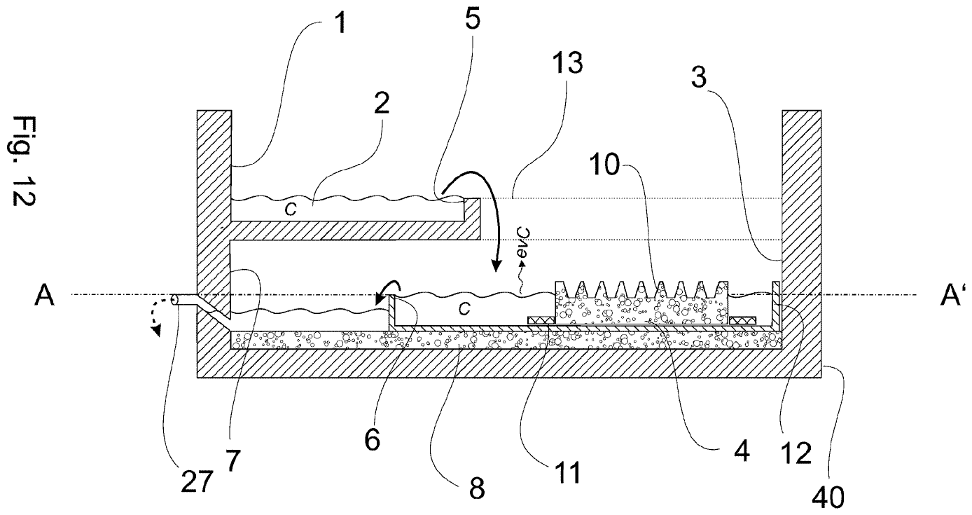

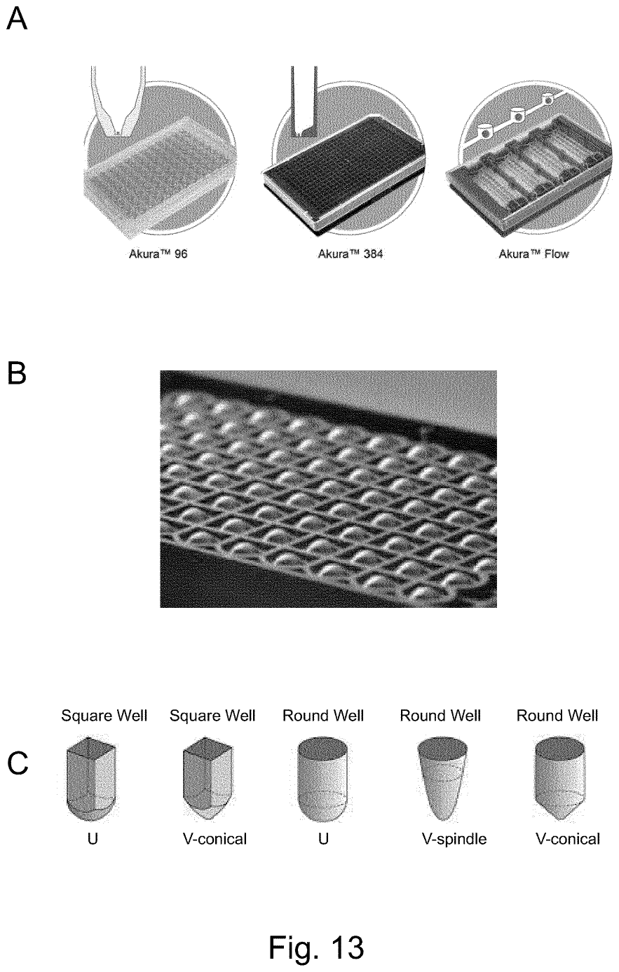

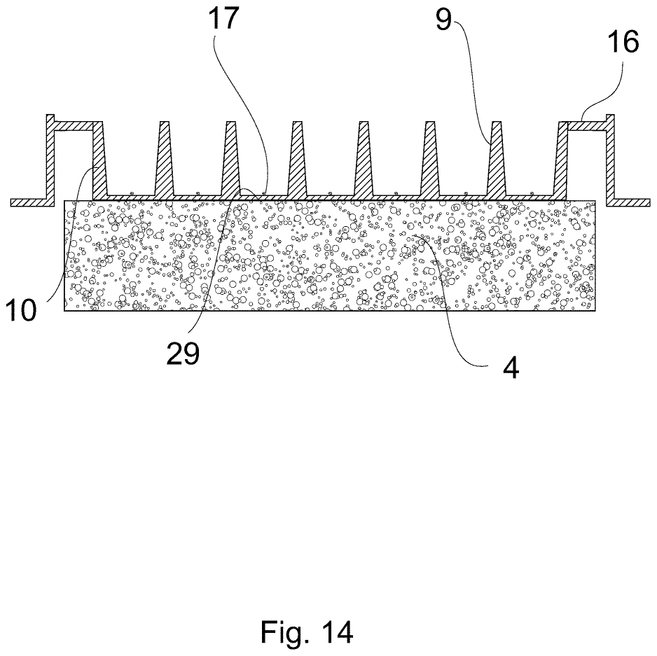

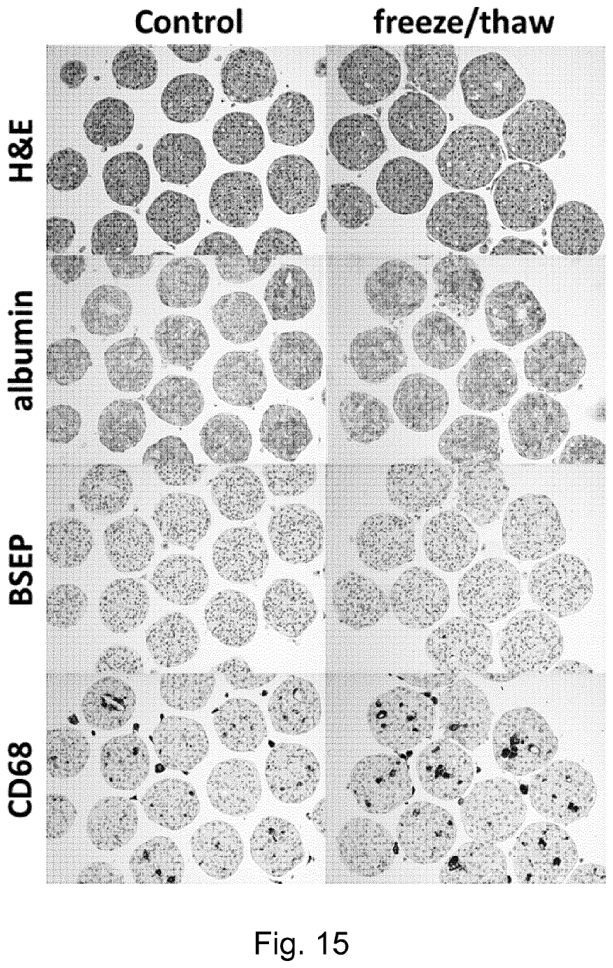

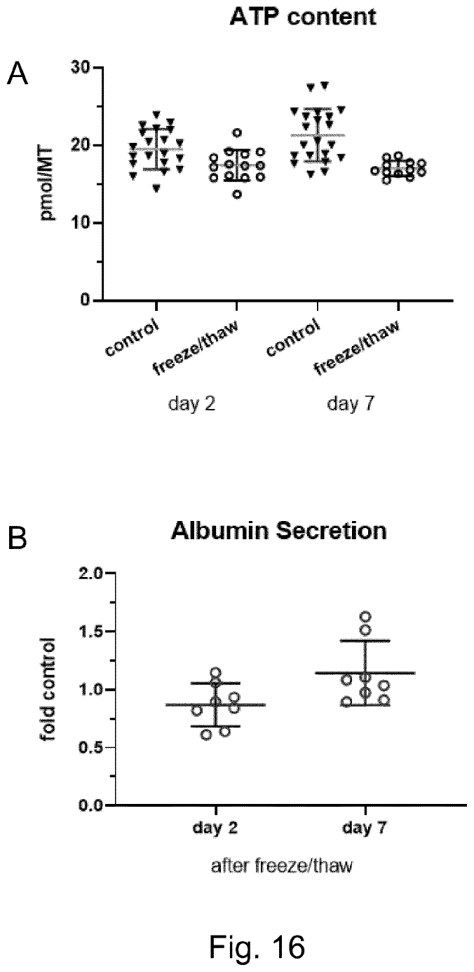

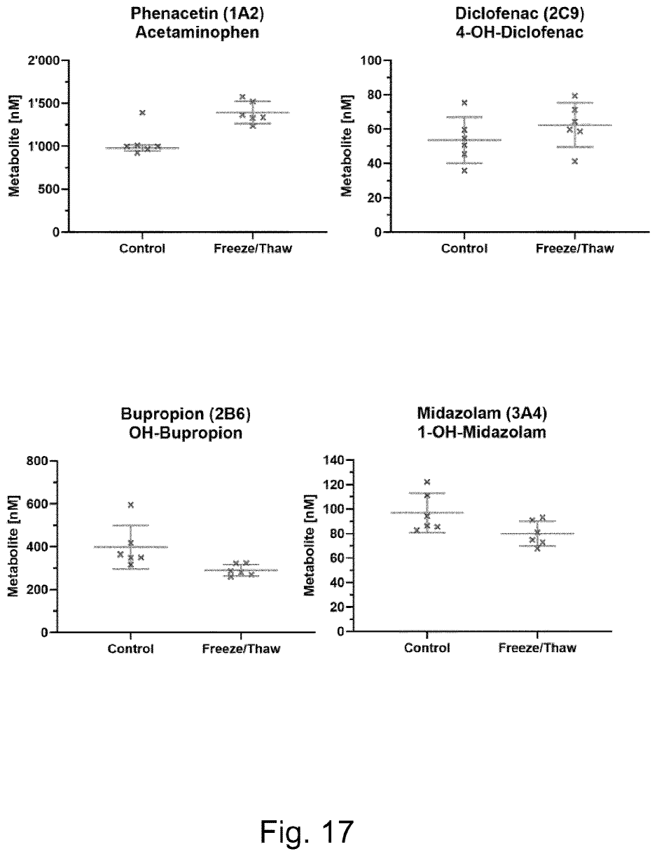

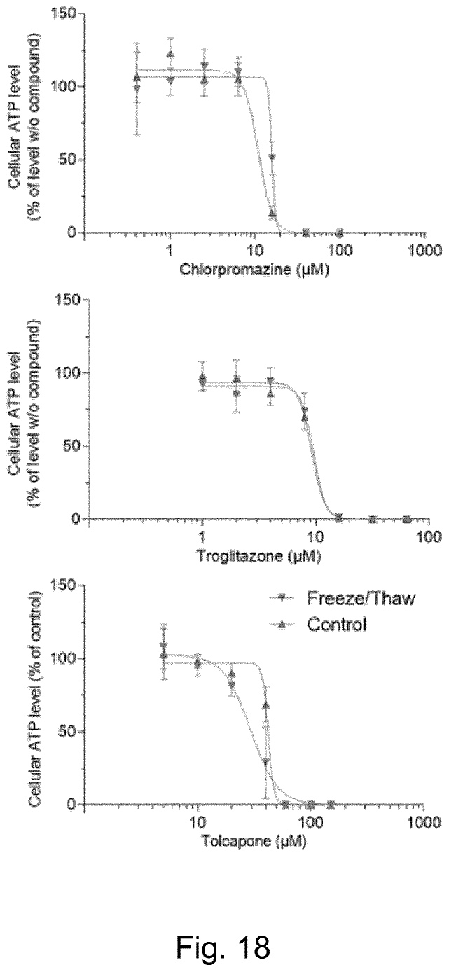

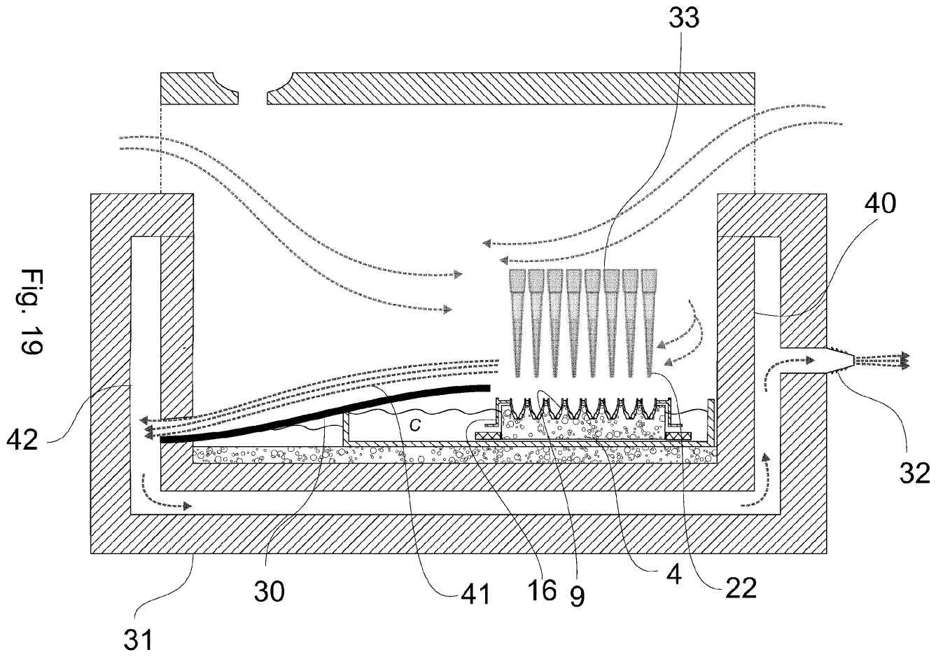

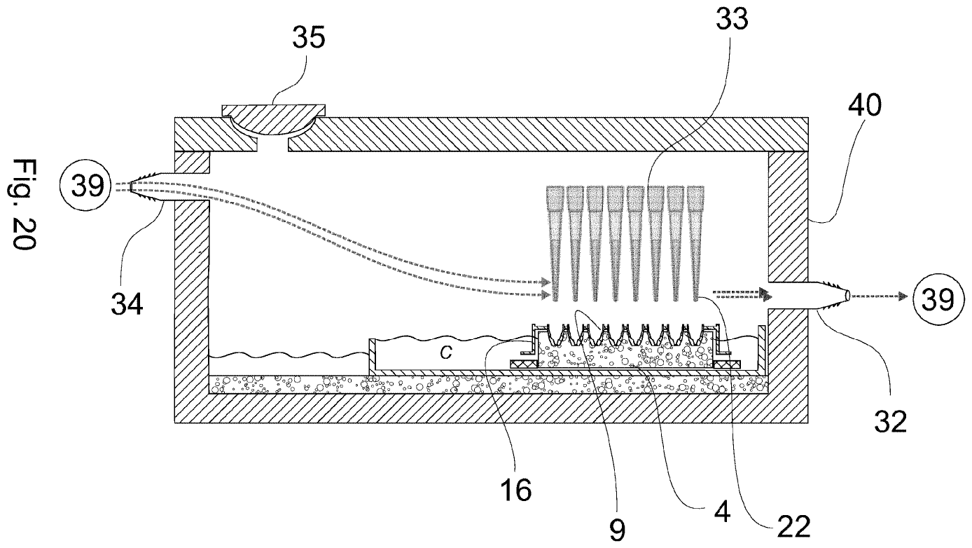

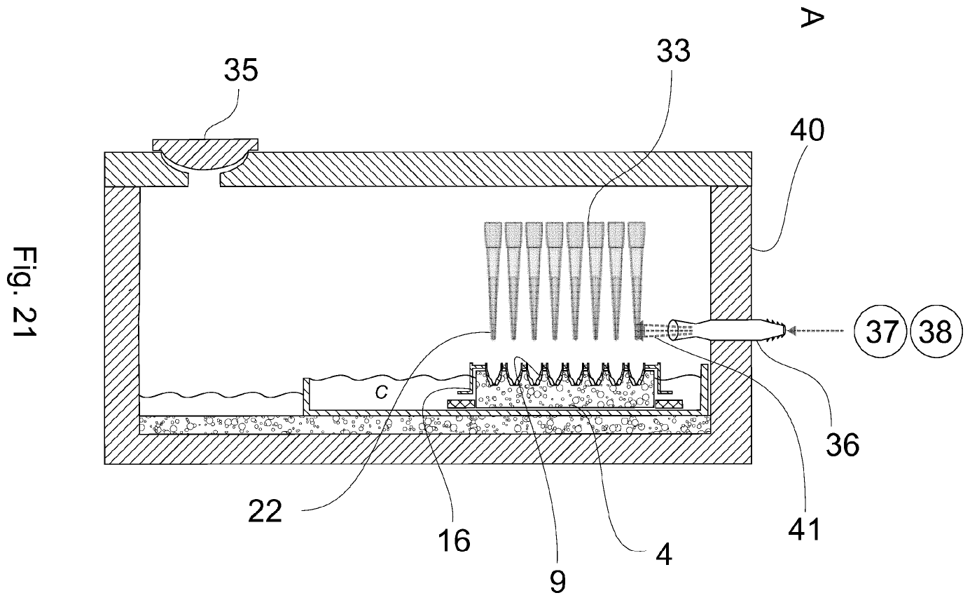

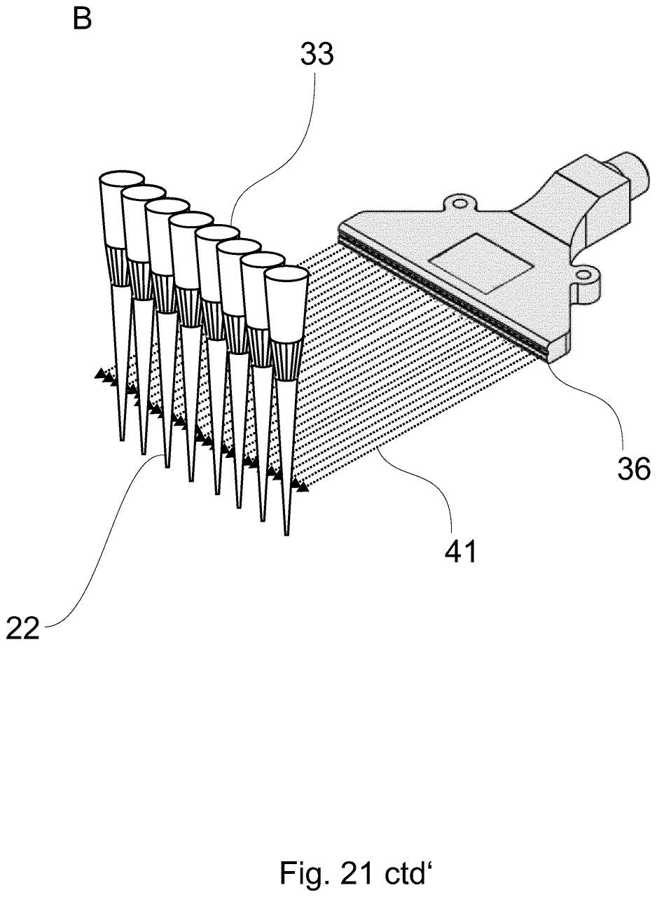

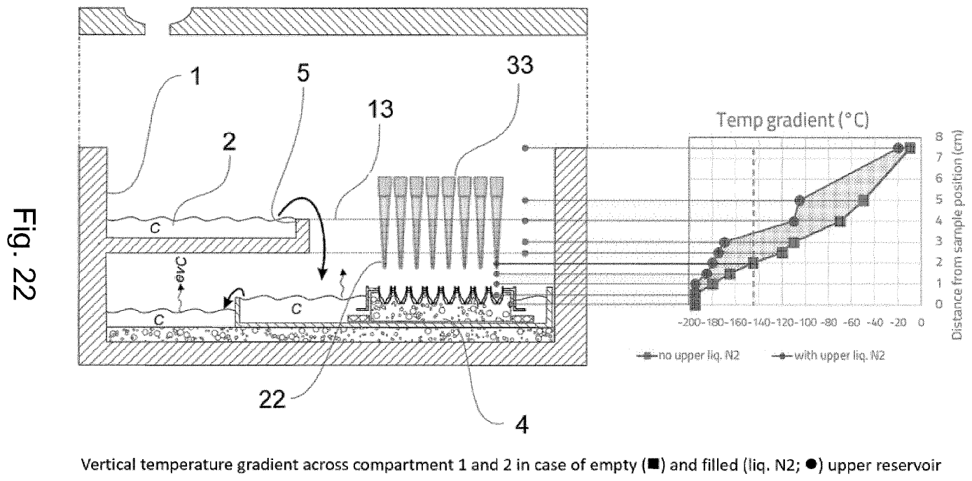

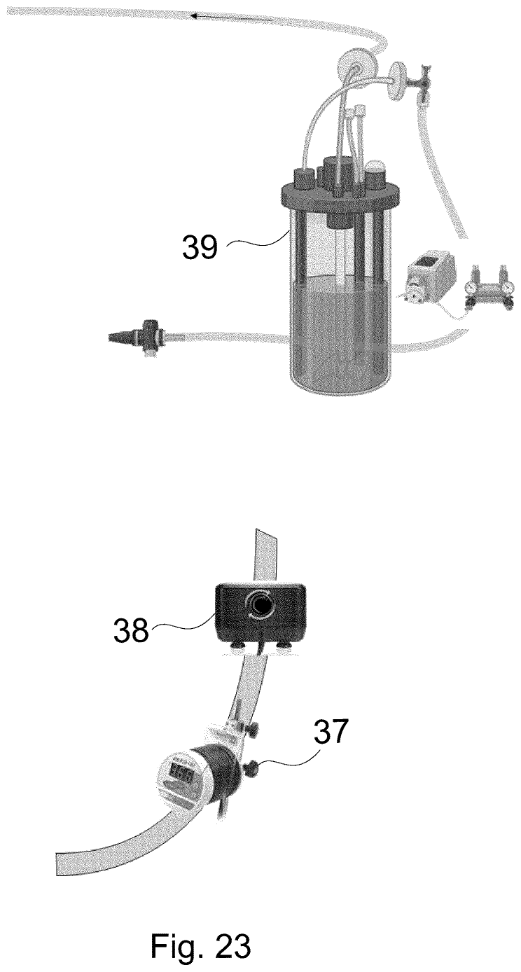

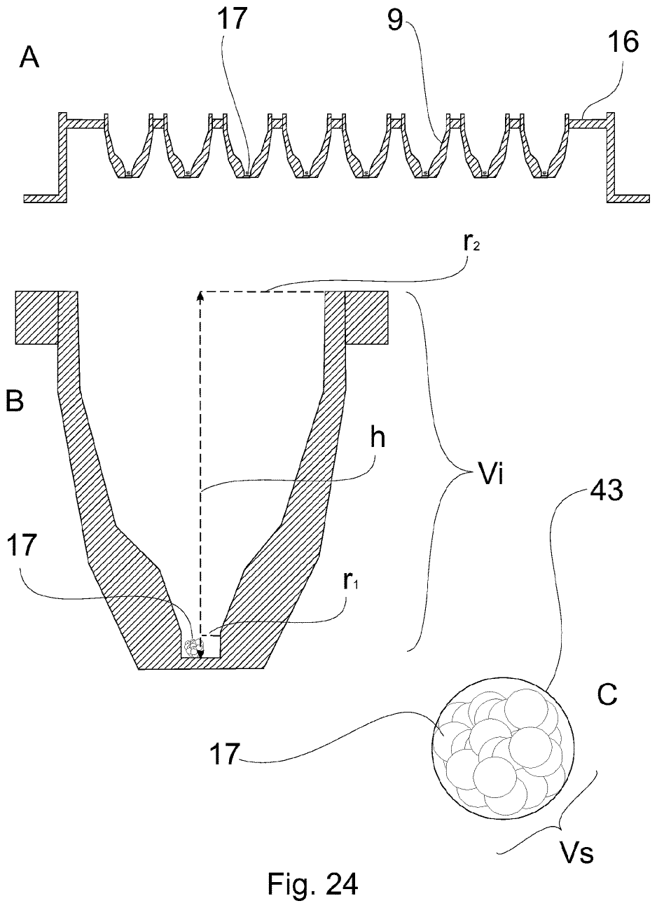

FIG. 1 C shows a lateral cross section of another system for freezing and/or vitrifying biological specimen according to the invention in a schematized and simplified form. The system comprises a first compartment 1 and a second compartment 3. The first compartment comprises an upper reservoir 2, and the second compartment 3 comprises a lower reservoir 12. Each reservoir is suitable and/or arranged to accommodate a cryogenic liquid C. The second compartment 3 is arranged below the first compartment 1. The second compartment comprises a support 4 for a recipient vessel. The support 4 for a recipient vessel comprises cavities 10 to accommodate wells or vials of the recipient vessel. The first compartment comprises an overflow rim 5 that allows cryogenic liquid C (see arrow) to flow from the upper reservoir 2 into the lower reservoir 12 of the second compartment 3. Cryogenic liquid C in the second compartment 3 may evaporate evC and hence establish a zone of cold air that cools the support 4, and the recipient vessel and its contents disposed thereon, from above. Cryogenic liquid C in the lower reservoir 12 of the second compartment 3 bathes the support 4, and the recipient vessel 16 and its contents disposed thereon, from sideways.

FIG. 1 D shows a lateral cross section of another system for freezing and/or vitrifying biological specimen according to the invention similar to the system shown in FIG. 1A. Contrary thereto, the support 4 for a recipient vessel is disposed on spacers 24, to allow the cryogenic liquid C in the lower reservoir 12 of the second compartment 3 to bath the support 4, and the recipient vessel and its contents disposed thereon, from sideways and from underneath.

FIG. 2 shows a lateral cross section of another system for freezing and/or vitrifying biological specimen according to the invention similar to the system shown in FIG. 1 B, yet with higher complexity. Contrary thereto, the support 4 for a recipient vessel is not necessarily disposed on spacers, but comprises a material that has an open-cell sponge-like structure and maintains elastic deformability at low temperatures (e.g., −70 °C or lower). In such way, it exerts a wicking effect on the cryogenic liquid. Accordingly, cryogenic liquid C in the lower reservoir 12 of the second compartment 3 is soaked into the support 4, and the recipient vessel and its contents disposed thereon are cooled from sideways and from underneath.

The system further comprises, as an optional feature, a cover lid 14 comprising an opening 15 suitable and/or arranged for filling in a cryogenic liquid into the upper reservoir 2 of the first compartment 1.

The system further comprises, as an optional feature, a side section 13 of the upper reservoir 2, which is shown in dotted lines, and hence forms a U-shaped or fully circumferential gallery of the upper reservoir 2.

The system further comprises, as an optional feature, a recess 28 in the overflow rim 5 of the first compartment, that allows cryogenic liquid C (see arrow) to flow from the upper reservoir 2 into the lower reservoir 12 of the second compartment 3.

The system further comprises, as an optional feature, a positioning grid 11 which allows secure and reproducible positioning of the support 4 for a recipient vessel.

As an optional feature, the second compartment 3 comprises a second overflow rim 6 which creates, within the second compartment 3, an overflow compartment 7. The second overflow rim 6 is arranged to allow the cryogenic liquid to flow from the lower reservoir 12 within the second compartment into the overflow compartment 7 if the level height of the cryogenic liquid in the lower reservoir 12 exceeds the height of the second overflow rim 6. For that purpose, the height of the second overflow rim 6 aligns with the height of the support 4 for the recipient vessel.

The system further comprises, as an optional feature, a third compartment 8 arranged below the second compartment 3. The third compartment 8 is in fluid communication with the second compartment 3 and/or the overflow compartment 7, and is suitable and/or arranged to accommodate a cryogenic liquid.

The third compartment 8 may comprise a material that has an open-cell sponge-like structure and maintains elastic deformability at low temperatures. In such way, it exerts a wicking effect on the cryogenic liquid. Accordingly, cryogenic liquid C in the lower reservoir 12 of the second compartment 3 is soaked into the third compartment 8 and into the support 4, and the recipient vessel and its contents disposed thereon are cooled from sideways and from underneath.

The system further comprises, as an optional feature, a filling nozzle 25 for cryogenic liquid suitable and/or arranged for filling in a cryogenic liquid into the lower reservoir 12 of the second compartment 2.

FIG. 3 shows a frontal cross section of a system for freezing and/or vitrifying biological specimen according to the invention, similar to the system shown in FIG. 2. It can be seen that the side section 13 of the upper reservoir 2 forms a U-shaped gallery of the upper reservoir 2.

FIG. 4 shows a perspective view of a system for freezing and/or vitrifying biological specimen according to the invention, similar to the system shown in FIG. 2. The opening 15 suitable and/or arranged for filling in a cryogenic liquid into the upper reservoir 2 of the first compartment 1. is clearly visible. It can be seen that the side section 13 of the upper reservoir 2 forms a U-shaped gallery of the upper reservoir 2 (the gallery can yet also be fully circumferential). The third compartment 8 which is in fluid communication with the second compartment 3 and/or the overflow compartment 7 is also clearly visible, as is the positioning grid 11 which allows secure and reproducible positioning of the support 4 for a recipient vessel.

FIG. 5 shows a perspective view of the support 4, and the recipient vessel 16 disposed thereon. The recipient vessel 16 adopts the format of a microtiter plate. The support 4 comprises a material that has an open-cell sponge-like structure and maintains elastic deformability at low temperatures. In such way, it exerts a wicking effect on the cryogenic liquid. Accordingly, cryogenic liquid C in the lower reservoir 12 of the second compartment 3 is soaked into the support 4, and the recipient vessel and its contents disposed thereon are cooled from sideways and from underneath. The cavities of the support 4 are arranged to be essentially form-locking with at least part of the wells of the recipient vessel. In such way, a direct contact of the wells with the cryogenic liquid is established.

FIG. 6 A shows a cross section of the support 4, and the recipient vessel 16 disposed thereon. Biological specimen 17 are provided within the wells 9 of the recipient vessel 16. The support 4 comprises a material that has an open-cell sponge-like structure and maintains elastic deformability at low temperatures. In such way, it exerts a wicking effect on the cryogenic liquid. The cavities 10 of the support 4 are arranged to be essentially form-locking with at least part of the wells of the recipient vessel. In such way, a direct contact of the wells with the cryogenic liquid is established.

FIG. 6 B shows a cross section of a recipient vessel 16 with wells 9 in which frozen and/or vitrified biological specimen 17 are provided. The recipient vessel is arranged on a vent plate 18 for thawing of biological specimen comprised the recipient vessel.

The vent plate 18 comprises cavities 19 on its upper side that are suitable and/or arranged to accommodate the wells 9 of the recipient vessel 16. The vent plate further comprises at least one supply channel suitable and/or arranged to supply a thawing medium to the outer walls of the wells 9 of the recipient vessel 16. The vent plate further comprises at least one exhaust channel 21 suitable and/or arranged to drain off thawing medium from the outer walls of the wells or vials of the recipient vessel.

FIG. 7 shows a cross section of a details view of a system for thawing of biological specimen 17 comprised in wells 9 of a recipient vessel 16. The system comprises a vent plate 18 as described in FIG. 6, and pipetting means 22 suitable and/or arranged for dispensing, into the wells 9 of the recipient vessel 16, a thawing medium 23.

FIGS. 8 A and B show a perspective view of the vent plate shown in FIG. 6 B.

FIG. 9 shows an embodiment of a system for freezing and/or vitrifying biological specimen according to the invention in three different steps of the freezing process. A pusher 26 is used to pump cryogenic liquid into the system, which then bathes the support 4, and the recipient vessel and its contents disposed thereon, from sideways. Also, cryogenic liquid is filled into a first compartment 1, which comprises an overflow rim 5 that allows cryogenic liquid) to flow from the upper reservoir 2 into the lower reservoir of the second compartment 3. As can be seen, the upper reservoir 2 comprises a side section 13 which forms a U-shaped gallery of the upper reservoir 2.

FIG. 10 (A) shows measurements of intracellular ATP in HepG2 Microtissues shortly after freeze/thaw and 2 days thereafter in comparison to non-frozen/thawed control microtissues. FIG. 10 (B) shows immunofluorescence images and corresponding GFP signal quantification data (FIG. 10 (C)) identifying GFP-expressing HepG2 Microtissues 2 days after freeze/thaw (vitrification) in comparison to non-frozen control tissues. Data demonstrate that neither viability, morphology nor growth rate was negatively affected by the vitrification and thawing process.

FIG. 11 shows a measured temperature gradient starting from above liquid nitrogen level in the second compartment and reaching into the first compartment, while distance above liq. nitrogen levels is indicated in millimeters (mm). The red dotted line indicates the temperature gradient achieved in presence of the upper reservoir, filled with liquid nitrogen, while the yellow line indicates the temperature gradient in the absence of an upper reservoir. The arrangement of an upper reservoir filled with liquid nitrogen stabilizes the temperature in the second compartment up to a height of 30-45 mm above liquid nitrogen level and warrants a 70-80 °C lower temperature up to a height of 105 mm.

FIG. 12 shows a lateral cross section of a system for freezing and/or vitrifying biological specimen according to the invention similar to the system shown in FIG. 2. The system further comprises a control nozzle 27 with an opening, wherein the height of the opening of the control nozzle aligns with the height of the second overflow rim of the second compartment. In such way, the cryogenic liquid is allowed to flow from the overflow compartment into and out of the control nozzle if the level height of the cryogenic liquid in the overflow compartment exceeds the height of the second overflow rim. The line A-A′ marks the height of the second overflow rim, the height of the support for the recipient vessel and the height of the opening of the control nozzle.

FIG. 13 shows the shapes of wells of microtiter plates that are used for cultivation of 3D cell cultures, with a conical or tapered cross section (FIG. 13 A, Akura™ 3D Microplates” provided by Insphero) and semi-spherical cross section (FIG. 13 B, “Spheroid Microplates” provided by Corning®). FIG. 13 C shows other possible shapes of wells of microtiter plates. In any case, the skilled person as aware how form locking between the one or more cavities of the support and the wells or vials of the recipient vessel can be accomplished, by properly shaping the cavities of the support.

FIG. 14 shows another embodiment in which the recipient vessel 16, which is provided in the form of a microtiter plate, comprises wells 9 yet has a flat bottom 29. In this case, the support 4, which optionally comprises a material that has an open-cell sponge-like structure and maintains elastic deformability at low temperatures, does not have any cavities 10, yet is float so as to be form locking with the flat bottom 29 of the recipient vessel. In such way, a direct contact of the wells 9 with the cryogenic liquid is established.

FIG. 15 shows results of hematoxylin and eosin (H&E) staining as well as immuno-staining, Albumin (Hepatocytes), BSEP (canalicular structures) and CD68 (Kupffer cells) in microtissues freeze/thawed according to the invention, relative to non-frozen microtissues (control).

FIG. 16 shows ATP content and albumin secretion of microtissues freeze/thawed according to the invention, relative to non-frozen microtissues.

FIG. 17 shows CYP450 activities (1A2, 2B6, 2C9, 3A4) of microtissues freeze/thawed according to the invention, relative to non-frozen microtissues (control). The CYP450 activities were assessed by exposure to prototypic substrates for 24 hours and quantification of respective metabolites by LC-MS.

FIG. 18 shows dose-response toxicity study (ATP) for Chlorpromazine, Troglitazone and Tolcapone of microtissues freeze/thawed according to the invention, relative to non-frozen microtissues (control).

FIG. 19 shows a lateral cross section of another system for freezing and/or vitrifying biological specimen according to the invention in a schematized and simplified form. The system comprises a pipette array 33 with tips 22 for dispensing biological specimen into one or more wells or vials 9 of the recipient vessel 16 arranged on the support 4.

The system further comprises a ventilation system to avoid rapid freezing of the liquid samples comprising the biological specimens within the pipette tips 22 at the moment of dispensing the latter into the wells or vials 9 of the recipient vessel 16. The ventilation system comprises
an outer hull 31 so that a hollow room 42 is formed between encasement 40 and outer hull 32.
a suction port 32 to draw cold gas from the hollow room 42 by means of a respective pump (not shown), and
a deflector plate 30 which is capable of establishing a non-turbulent flow of gas like e.g., air, which bathes the pipette tips only, yet does not create any turbulences which could affect the overall temperature regimen within the system, including the “Kaltluftsee”.

FIG. 20 shows a lateral cross section of another system for freezing and/or vitrifying biological specimen according to the invention in a schematized and simplified form. The system comprises a pipette array 33 with tips 22 for dispensing biological specimen into one or more wells or vials 9 of the recipient vessel 16 arranged on the support 4.

The system further comprises a ventilation system to avoid rapid freezing of the liquid samples comprising the biological specimens within the pipette tips 22 at the moment of dispensing the latter into the wells or vials 9 of the recipient vessel 16. The ventilation system comprises
a suction port 32 to draw cold gas (like e.g. cold air) from the system by means of a warm air source (39, see FIG. 23), and
an input port (34) for warm gas (like e.g. warm air) provided by a warm air source (39).

FIG. 21A shows a lateral cross section of another system for freezing and/or vitrifying biological specimen according to the invention in a schematized and simplified form. The system comprises a pipette array 33 with tips 22 for dispensing biological specimen into one or more wells or vials 9 of the recipient vessel 16 arranged on the support 4.

The system further comprises a ventilation system to avoid rapid freezing of the liquid samples comprising the biological specimens within the pipette tips 22 at the moment of dispensing the latter into the wells or vials 9 of the recipient vessel 16. The ventilation system comprises a flat air nozzle 36 connected to a tube heating device 37 and an air pump 38 (both shown in FIG. 23).

Such flat air nozzle 36 is capable of producing a laminar flow 41 of warm gas, like e.g., air, hence ensuring that the latter bathes the pipette tips 22 of the pipette array only, yet does not create any turbulences which could affect the overall temperature regimen within the system, including the “Kaltluftsee”.

FIG. 21B shows an example of such flat air nozzle 36, and the laminar flow 41 of warm gas, like e.g., air, it creates, which bathes the pipette tips 22 of the pipette array 33 only.

FIG. 22 shows a vertical temperature gradient across first and second compartment wherein the upper reservoir is either empty (▪) or filled with liquid N2 filled (●) as measured with a temperature probe.

FIG. 23 shows, schematically and exemplarily,
a warm air source 39, which comprises a warm water washing bottle and a gas tubing connected to a pump, so that cold air is drawn from the suction port 32 of the system shown in FIG. 20, warmed, and resupplied to the input port 34 of said system, and
a tube heating device 37 and an air pump 38 to be connected to the flat air nozzle 36 as referred to in FIG. 21.

FIG. 24 A shows a recipient vessel 16 comprising wells or vials 9, each of which comprises one frozen or vitrified biological specimen 17. The wells or vials comprise an inner volume Vᵢ that is at least 100× greater than the volume Vₛ of the biological specimen 17, including surrounding vitrified or frozen storage or culture medium, if present.

FIG. 24 B shows an enlarged detail of FIG. 24 A. The well or vial 9 has a tapered or conical shape. Its volume can be approximated by the formula V=(⅓)·π·h·(r₁²+r₂²+(r₁·r₂)), in which h is the height of the well or vial, r₁ is the lower radius of the vial and r₂ is the upper radius of the vial.

FIG. 24 C shows an example of a frozen or vitrified biological specimen 17 including surrounding vitrified or frozen storage or culture medium 43. The volume Vₛ is approximated by the formula V=4/3·π·r³, in which r is the radius of the biological specimen 17 including surrounding vitrified or frozen storage or culture medium 43.

### DETAILED DESCRIPTION

According to a first aspect of the invention, a system for freezing and/or vitrifying biological specimen is provided. The system comprises an encasement (40) arranged to accommodate a cryogenic liquid (C), and a support (4) for a recipient vessel (16) suitable and/or arranged for accommodating one or more biological specimen.

According to embodiments of the system, the support (4) is a support according to the below description.

According to one embodiment, the system comprises a first compartment (1) and a second compartment (3). The first compartment comprises an upper reservoir (2), and the second compartment comprises a lower reservoir (12). Each reservoir is suitable and/or arranged to accommodate a cryogenic liquid (C), The second compartment is arranged below the first compartment, and the second compartment comprises a support (4) for a recipient vessel suitable and/or arranged for accommodating one or more biological specimen.

Such system allows rapid freezing and/or vitrification of biological specimen without formation of ice crystals that could harm or affect the specimen. Further, it avoids to directly apply cryogenic liquid (C) to the biological specimen. Such direct contact is commonly hampered by difficulties to accurately dose the cryogenic liquid (C), and by splashing provoked by the Leidenfrost effect once the cryogenic liquid (C) contacts warmer surfaces. Furthermore, such system avoids the direct pipetting of cryogenic liquid (C) into the vials comprising the biological specimen—which is difficult to control due to the effect that cryogenic liquid is squeezed out of the pipette tips by evaporating cryogenic liquid in the pipette.

Regarding embodiments of the support, system and/or method described elsewhere herein, the features, characteristics and advantages discussed in connection with said embodiments, as well as the embodiments as such, are understood to apply to the system and its embodiments discussed above and in the following, even if they are not repeated again.

According to one embodiment of the system according to the invention, the system further comprises a recipient vessel (16) suitable and/or arranged for accommodating one or more biological specimen.

According to one embodiment of the system according to the invention, the upper reservoir (2) comprises a first overflow rim (5) arranged to allow the cryogenic liquid to flow into the lower reservoir (12) if the level height of the cryogenic liquid in the upper reservoir (2) exceeds the height of the first overflow rim (5).

This arrangement ensures that the support for the recipient vessel is always fully bathed in croygenic liquid, and that a zone of cold vapour (“Kaltluftsee”) establishes above the croyogenic liquid that still cools everything underneath, including the support and the recipient vessel and ist content arranged thereon.

According to one embodiment of the system according to the invention, the second compartment (3) comprises a second overflow rim (6) arranged to create, within the second compartment (3), an overflow compartment (7), wherein the second overflow rim (6) is arranged to allow the cryogenic liquid to flow from the lower reservoir (12) within the second compartment into the overflow compartment (7) if the level height of the cryogenic liquid in the lower reservoir (12) exceeds the height of the second overflow rim (6).

According to one embodiment of the system according to the invention, the height of the second overflow rim (6) aligns with the height of the support (4) for the recipient vessel (16). In such way, it is made sure that the wells or vials in the recipient vessel, when disposed on the support, are not flooded with cryogenic liquid.

According to one embodiment of the system according to the invention, the system further comprises a third compartment (8) arranged below the second compartment (3), which third compartment (8) is in fluid communication with the second compartment (3) and/or the overflow compartment (7), and is suitable and/or arranged to accommodate a cryogenic liquid.

According to one embodiment of the system according to the invention, the system further comprises a cover lid (14) comprising an opening (15) suitable and/or arranged for filling in a cryogenic liquid (C) into the upper reservoir (2) of the first compartment (1).

According to one embodiment of the system according to the invention, the system further comprises a filling nozzle (25) for the cryogenic liquid which is suitable and/or arranged for filling the cryogenic liquid directly into the second compartment (3) and/or the overflow compartment (7) and/or into the third compartment (8).

According to one embodiment of the system according to the invention, the system further comprises a control nozzle (27) with an opening, wherein the height of the opening of the control nozzle aligns with the height of the second overflow rim (6) of the second compartment (3)

In such way, the cryogenic liquid is allowed to flow from the overflow compartment (7) into and out of the control nozzle (27) if the level height of the cryogenic liquid in the overflow compartment (7) exceeds the height of the second overflow rim (6).

According to one embodiment of the system according to the invention, the support (4) for the recipient vessel (16) comprises one or more cavities (10) on its upper side that are suitable and/or arranged to accommodate the one or more wells or vials (9) of the recipient vessel (16) suitable and/or arranged for accommodating biological specimen.

According to one embodiment of the system according to the invention, the one or more cavities (10) are arranged to be essentially form-locking with at least part of the one or more wells or vials (9) of the recipient vessel (16).

According to one embodiment of the system according to the invention, wherein the support (4) for the recipient vessel comprises a material that has a wicking effect on the cryogenic liquid.

In such way, it is awarded that cryogenic liquid is directed quickly and directly to the wells or vials (9) of the recipient vessel (16). This enables a quick and uninterrupted cooling of the biological specimen provided therein.

According to one embodiment of the system according to the invention, wherein the support (4) for the recipient vessel comprises a material that has an open-cell sponge-like structure.

According to one embodiment of the system according to the invention, the support (4) for the recipient vessel comprises a material that maintains elastic deformability at a temperature below −70 °C and/or in the presence of the cryogenic liquid (C).

In such way, a permanent close contact between the one or more cavities (10) of the support (4) and the wells or vials (9) of the recipient vessel (16) is awarded even under cryogenic conditions. This enables a quick and uninterrupted cooling of the biological specimen provided therein.

According to one embodiment of the system according to the invention, the support (4) for the recipient vessel has an absolute height of between ≥5 and ≤50 mm.

In several embodiments, the support (4) for the recipient vessel has an absolute height of between ≥10 and ≤40 mm, between ≥15 and ≤30 mm, or 20 mm+/−3 mm.

According to one embodiment of the system according to the invention, the support (4) for the recipient vessel comprises a material selected from the group consisting of
melamine (1,3,5-Triazine-2,4,6-triamine) or melamine resin,
polyurethane, and/or
cross linked polyolefin foam

The inventors have surprisingly shown that these materials
a) have a wicking effect on cryogenic liquids, as discussed above, and
b) maintain elastic deformability at a temperature below −70 °C and/or in the presence of the cryogenic liquid.

As discussed above both features enable a quick and uninterrupted cooling of the biological specimen provided therein.

GB2377985A discloses a disposable container for refrigerated animal/human tissue or fluid. The device comprises a liquid refrigerant (liquid nitrogen) absorbent material having one or more pockets for receiving containers for biological specimen so that the refrigerant is in close proximity of the containers.

The liquid absorbent material is preferably a foamed material or sponge, and preferably a foam commonly known as Oasis®, which is normally used by florists as a base for flower arrangements. GB2377985A mentions that this material would be particularly advantageous because, once pockets have been formed, the latter will retain their shape. This means, in turn, that, different to the material that is preferred in the present invention, the foam material of GB2377985A is plastically deformable (i.e., once deformed, it retains its shape), and is not elastically deformable.

The inventors of the present invention have experimentally confirmed that the Oasis® material disclosed in GB2377985A as preferred is not elastically deformable neither at room temperature nor under cryogenic conditions (e.g., at −196 °C). This means that, contrary to the respective embodiment of the present invention, such material does not award a permanent close contact between the cavities (“pockets”) of the support and the wells or vials (“containers”) of the recipient vessel, and therefore does not enable a quick and uninterrupted cooling of the biological specimen provided therein.

The inventors of the present invention have furthermore experimentally confirmed that for example melamine (1,3,5-Triazine-2,4,6-triamine) or melamine resin, does indeed
a) have a wicking effect on cryogenic liquids, and
b) maintains elastic deformability at a temperature below −70 °C and/or in the presence of the cryogenic liquid.

According to one embodiment of the system according to the invention, the third compartment (8) comprises a material that has at least one of the following properties:
a) a wicking effect on the cryogenic liquid,
b) an open-cell sponge-like structure,
c) maintains elastic deformability at a temperature below −70 °C and/or in the presence of the cryogenic liquid, and/or
d) comprises a material selected from the group consisting of
   melamine (1,3,5-Triazine-2,4,6-triamine) or melamine resin,
   polyurethane, and/or
   cross linked polyolefin foam

According to one embodiment of the system according to the invention, the cryogenic liquid is selected from the group consisting of
liquid nitrogen,
liquid helium,
liquid argon,
liquid oxygen,
a mixture of dry ice (CO₂) and an organic solvent.

Preferably the organic solvent mixed with dry ice is acetone and/or ethanol.

According to one embodiment of the system according to the invention, the support (4) for the recipient vessel is arranged on one or more spacers (24). In such way, the cryogenic liquid can bathe the support also from underneath.

According to one embodiment of the system according to the invention, the recipient vessel (16) comprises one or more wells or vials (9) suitable and/or arranged for accommodating biological specimen.

According to one embodiment of the system according to the invention, the recipient vessel (16) is a microtiter plate or an array of vials corresponding to a 6, 24, 96, or 384 microtiter plate layout.

As discussed herein, the one or more cavities (10) of the support (4) are preferably arranged to be essentially form-locking with at least part of the one or more wells or vials (9) of the recipient vessel (16).

Different types of microtiter plates can be used in the context of the present invention, including regular microtiter plates, comprising tapered, conical, cylindrical or semi-spherical wells, as well as plates specifically made for cultivation of microspheres, as discussed in Larsen B (2015), the content of which is incorporated by reference herein for enablement purposes.

Such plates may comprise more sophistically shaped wells, like e.g. “Akura™ 3D Microplates” provided by Insphero, having a tapered cross section (see FIG. 13 A), or “Spheroid Microplates” provided by Corning® (see FIG. 13 B).

The form locking principle between the one or more cavities (10) of the support (4) and the wells or vials (9) of the recipient vessel (16) is shown in FIG. 6A. The skilled person is able to transfer this principle to other recipient vessels having different shapes of their wells or vials (see FIG. 13), or even to embodiments wherein the recipient vessel has a flat bottom (see FIG. 14).

According to another embodiment of the invention, the system further comprises pipetting means (22, 33) for dispensing biological specimen (17) into one or more wells or vials (9) of the recipient vessel (16).

According to another embodiment of the invention, the system further comprises ventilation means (30, 32, 34, 36, 37, 38, 39) to generate a warm gas current along the tips (22) of the pipetting means (33).

Such ventilation means may optionally comprise at least one element selected from the group consisting of
deflector plate (30)
air suction port (32)
air input port (34)
flat air nozzle (36)
tube heating device (37)
air pump (38)
warm air source (39)

Such ventilation means avoid rapid freezing of the liquid sample comprising the biological specimen (17) within the pipette tips (22) at the moment of dispensing the latter into the wells or vials (9) of the recipient vessel (16). It is important to make sure that such effect is kept locally confined, as e.g. shown in the figures.

Such flat air nozzle (36) is capable of producing a laminar flow (41) of warm gas, like e.g., air, hence ensuring that the latter bathes the pipette tips only, yet does not create any turbulences which could affect the overall temperature regimen within the system, including the “Kaltluftsee”.

A similar effect is achieved by the deflector plate (30), which creates a non-turbulent flow of gas (41), like e.g. air.

Further, the preferred embodiments and their characteristics discussed elsewhere herein regarding apply mutatis mutandis to this aspect of the invention, i.e., the system

According to another aspect of the invention, a support (4) for freezing or vitrifying of biological specimen comprised in a recipient vessel (16) is provided. The support comprises a material that has a wicking effect on a cryogenic liquid.

According to one embodiment of the support according to the invention, the cryogenic liquid is selected from the group consisting of
liquid nitrogen,
liquid helium,
liquid argon,
liquid oxygen,
a mixture of dry ice (CO₂) and an organic solvent.

The following table shows boiling points of suitable cryogenic liquids.

| type | Boiling pint at normal pressure of about |
|---|---|
| liquid nitrogen | −196 °C |
| liquid helium | −269 °C |
| liquid argon | −186 °C |
| liquid oxygen | −183 °C |
| mixture of dry ice (CO₂) and an | −72 °C (with ethanol) |
| organic solvent | |

Preferably the organic solvent mixed with dry ice is acetone and/or ethanol.

According to one embodiment of the support according to the invention, the recipient vessel is suitable and/or arranged for accommodating one or more biological specimen.

According to one embodiment of the support according to the invention, the support comprises a material that has an open-cell sponge-like structure. In one embodiment, the support comprises an open-celled three-dimensional fiber network structure.

In one embodiment, the support comprises an open-celled three-dimensional fiber network structure.

According to one embodiment of the support according to the invention, the support comprises a material that maintains elastic deformability at a temperature below −70 °C and/or in the presence of the cryogenic liquid. In such way, a proper form locking with a recipient vessel is achieved over a large temperature range.

According to one embodiment of the support according to the invention, the support has an absolute height of between ≥5 and ≤50 mm.

In several embodiments, the support has an absolute height of between ≥10 and ≤40 mm, between ≥15 and ≤30 mm, or 20 mm+/−3 mm.

This height range has turned out practicable, as it satisfies two constraints, namely a) it is not too high to negatively affect the wicking effect that draws cryogenic liquid into the support and, b) it is high enough to allow a sufficiently high level of cryogenic liquid provided in a compartment that encompasses the support, without flooding the recipient vessel.

According to one embodiment of the support according to the invention, the support comprises a material selected from the group consisting of
melamine (1,3,5-Triazine-2,4,6-triamine) or melamine resin,
polyurethane, and/or
cross linked polyolefin foam

These materials meet the requirements set forth above as to wicking effect and elastic deformability at low temperatures.

Melamine and its resins are most commonly known for being used in dinnerware, laminate flooring, and dry erase boards. Melamine foam is used as insulation, soundproofing material and in polymeric cleaning products, such as the so called “Magic Eraser”.

The inventors surprisingly found that melamine and melamine resins, as a material used in the support according to the invention, have favourable properties, as the material
maintains elastic deformability at a temperature below −70 °C and/or in the presence of a cryogenic liquid
can be provided to have an open-cell sponge-like structure
and can be provided to have a wicking effect on a cryogenic liquid.

Cross-linked, polyolefin foams have been found suitable materials for cryogenic vessels. Several characteristics of polyolefin foams make them advantageous for use as cryogenic vessels: they can be fabricated easily into appropriate shapes by molding, casting or otherwise forming the foam from monomer or polymer, or by machining finished blocks; their cell structure is typically fine enough to contain, for example, liquid nitrogen, without leaking; they are durable enough to withstand repeated exposures to cryogenic temperatures; they are largely non-reactive; and they typically have both a low thermal conductivity and a low volumetric heat capacity. As used here, the term “polyolefin foam” refers to polyethylene foam, polypropylene foam, polyethylene-polypropylene mixtures and copolymers, and foams that contain a mixture or copolymer of olefin monomer and other monomers, to the extent that the mixed foams have at least some of the favourable characteristics noted above.

According to one embodiment of the support according to the invention, the support comprises one or more cavities (10) on its upper side that are suitable and/or arranged to accommodate one or more wells or vials (9) of a recipient vessel (16) suitable and/or arranged for accommodating biological specimen.

According to one embodiment of the support according to the invention, the one or more cavities (10) are arranged to be essentially form-locking with at least part of the one or more wells or vials (9). Such form-locking between the one or more cavities (10) of the support (4) and the wells or vials (9) of the recipient vessel (16). is shown in FIG. 6A. The skilled person is able to transfer this principle to other recipient vessels having different shapes of their wells or vials.

According to one embodiment of the support according to the invention, the recipient vessel (16) is a microtiter plate or an array of vials corresponding to a 6, 24, 96, or 384 microtiter plate layout.

According to another embodiment, the recipient vessel (16) comprises wells or vials (9) yet has a flat bottom (29). In this case, the support (4), which optionally comprises a material that has an open-cell sponge-like structure and maintains elastic deformability at low temperatures, does not have any cavities (10), yet is float so as to be form locking with the flat bottom (29) of the recipient vessel (16). In such way, a direct contact of the wells (9) with the cryogenic liquid is established.

According to another aspect of the invention, a vent plate (18) for thawing of biological specimen comprised in a recipient vessel (16) is provided. The vent plate comprises one or more cavities (19) on its upper side that are suitable and/or arranged to accommodate one or more wells or vials (9) of a recipient vessel suitable and/or arranged for accommodating biological specimen.

The vent plate further comprises
one or more supply channels (20) suitable and/or arranged to supply a thawing medium to the outer walls of the wells or vials (9) of the recipient vessel, and
one or more exhaust channels (21) suitable and/or arranged to drain off thawing medium from the outer walls of the wells or vials (9) of the recipient vessel.

According to one embodiment of the vent plate (18) according to the invention, the thawing medium (23) is a fluid, liquid or gas.

According to one embodiment, the thawing medium (23) is air.

According to one embodiment of the vent plate according to the invention, the thawing medium (23) comprises a temperature of between ≥−150 °C and ≤+100 °C.

According to several embodiments, the thawing medium comprises a temperature of between ≥4 °C and ≤+100 °C, between ≥15 °C and ≤+90 °C, and preferably between ≥20 °C and ≤+37 °C.

According to another aspect of the invention, a system for thawing of biological specimen comprised in a recipient vessel (16) is provided. The system comprises
a) a vent plate (18) according to the above description, and
b) pipetting means (22) suitable and/or arranged for dispensing, into the wells or vials (9) of the recipient vessel (16), a thawing medium (23).

According to one embodiment of the thawing system according to the invention, the thawing medium is a fluid, liquid or gas.

According to one embodiment of the thawing system according to the invention, the thawing medium used in the vent plate is a gas, preferably air, as discussed above, also with regard to suitable temperatures.

According to one embodiment of the thawing system according to the invention, the thawing medium used in the pipetting system is a liquid, preferably an aqueous liquid. According to several embodiments, the thawing medium used in the pipetting system comprises a temperature of between ≥4 °C and ≤+80 °C, between ≥15 °C and ≤+70 °C, and preferably between ≥20 °C and ≤+37 °C.

According to further embodiments of the system, vent plate (18) or support (4) according to the invention, the biological specimen is at least one selected from the group consisting of
one or more tissue samples,
one or more gametocytes (oocytes or spermatocyte),
one or more blastocysts,
one or more individual cells,
one or more cellular aggregates (e.g. microtissues, spheroid), and/or
one or more organoids derived from stem cells by self-organization and differentiation.

In one embodiment the biological specimen is comprised in a liquid, e.g., a storage or culture medium. In such case, the biological specimen is described to be comprised in a liquid sample.

According to one embodiment, the biological specimen does not involve a bioartificial scaffold, i.e., is scaffold-free. In one embodiment, the biological specimen is a microtissue, spheroid or organoid that is scaffold-free.

Such scaffold materials are for example disclosed in Carletti et al (2011), and include natural polymers (like e.g. Collagen, Chitosan, Glycosaminoglycans, Silk Fibroin, Agarose, Alginate, and Starch) and synthetic polymers (like e.g. Poly(glycolic acid), Poly(lactic acid), and copolymers, Poly(ε-caprolactone), Polyurethane, Poly(ortho ester), and Poly(anhydrides)). Such scaffold material is oftentimes used in the formation of microtissues, yet may negatively affect the results of assays in which the microtissues are used.

According to another aspect of the invention, a method for freezing and/or vitrifying biological specimen is provided. Said method comprises the steps of
a) placing a recipient vessel (16) suitable and/or arranged for accommodating one or more biological specimen on a support (4) according to the above description, the support being arranged in a system according to the above description
b) before or after step a), dispensing one or more biological specimen (17) into one or more wells or vials (9) of the recipient vessel, and
c) before or after step a) or b), filling a cryogenic liquid into the system according to the above description.

According to one embodiment, the method further comprises the step of adding one or more cryoprotective agents (CPA) to the biological specimen before step b).

According to one embodiment, the method comprises the successive equilibration of the biological specimen with 2 or 3 different increasing concentrations of CPAs.

In several embodiments, the cryoprotective agent comprises at least one of dimethyl sulfoxide (DMSO) and/or one or more of an alcohol, a sugar, an amides or imide, and/or a macromolecule, wherein the alcohol is selected from methanol, glycerol, sorbitol, ethylene glycol, propylene glycol, butylene glycol, inositol, xylitol, mannitol, adonitol, arabitol, ribitol, erythritol, perseitol, threitol, galactitol, pinitol, xylitol, and combinations thereof; and the sugar is selected from sucrose, trehalose, maltose, arabinose, lactose, mannose, xylose, galactose, fructose, glucose, dextran, melezitose, raffinose, nigerotriose, maltotriose, maltotriulose, kestose, cellobiose, chitobiose, lactulose, and combinations thereof, whereas amides and imides are selected of formamide, acetamide, propionamide, lactamide, butyramide, malonamide, and combinations thereof, and whereas macromolecules are selected of Ficoll, polyethylene glycol, polyvinylpyrrolidone, polvinylalcohol, hyaluronan, and combinations thereof. See in this regard Whaley et al (2021), the content of which is incorporated herein by reference for enablement purposes.

According to one embodiment, the dispensing one or more biological specimen (17) into one or more wells or vials (9) of the recipient vessel comprises the steps of
i) picking up the one or more biological specimen from a plate comprising biological specimen with one or more pipettes, or a pipette array (33) comprising one or more pipette tips (22),
ii) letting the one or more biological specimen sediment to the bottom of the one or more pipette tips (22), and
iii) dispensing the one or more biological specimen from the pipette tips (22) into the one or more wells or vials (9) of the recipient vessel by releasing a droplet comprising a biological specimen.

The biological specimen, even if sedimented, can optionally be surrounded by liquid medium.

According to one embodiment of that method, only one biological specimen (e.g., only one microtissue) is dispensed per well or vial (9) of the recipient vessel.

According to embodiments of that method the droplet comprising the biological specimen has a volume of between ≥0.05 μl and ≤8 μl. According to further embodiments of that method the droplet comprising the biological specimen has a volume of about 0.1 μl, about 0.2 μl, about 0.3 μl, about 0.4 μl, about 0.5 μl, about 0.6 μl, about 0.7 μl, about 0.8 μl, about 0.9 μl, about 1 μl, about 1.2 μl, about 1.4 μl, about 1.5 μl, about 1.6 μl, about 1.8 μl, about 2 μl, about 2.2 μl, about 2.4 μl, about 2.6 μl, about 2.8 μl, about 3 μl, about 3.2 μl, about 3.4 μl, about 3.6 μl, about 3.8 μl or about 4 μl.

Several embodiments of the present invention ensure that, during the dispensing step, the sample comprising the biological specimen does not freeze in the pipette tip (22), although the latter is brought close to the one or more wells or vials (9) of the recipient vessel, all of which are cooled down due to the presence of the cryogenic liquid. hence, only after the specimen is dispensed into its well or vial, the freezing/vitrification starts immediately.

According to another aspect of the invention, a method of storing biological specimen frozen and/or vitrified with a method according to the above description is provided. The method comprises the steps of
a) keeping the recipient vessel (16) which accommodates one or more frozen and/or vitrified biological specimen within the system according to the above description,
b) optionally, positioning a lid (14) on top of the system, and
c) optionally, replenishing cryogenic liquid into the system according to the above description.

According to another aspect of the invention, a method of thawing biological specimen frozen and/or vitrified with a method according to the above description is provided. The method comprises the steps of
a) removing the recipient vessel (16) from the support (4)
b) placing the recipient vessel (16) on a vent plate (18) according to the above description
c) supplying the vent plate (18) with a thawing medium
d) optionally, simultaneously with step c) or sequentially prior or after step c), dispensing a thawing medium from pipetting means (22) arranged above of the recipient vessel (16).

According to one embodiment of the method according to the invention, the thawing medium is a fluid, liquid or gas.

According to one embodiment of the method according to the invention, the thawing medium used in the vent plate is a gas, preferably air, as discussed above, also with regard to suitable temperatures.

According to one embodiment of the method according to the invention, the thawing medium used in the pipetting system is a liquid, preferably an aqueous liquid. According to several embodiments, the thawing medium used in the pipetting system comprises a temperature of between ≥4 °C and ≤+80 °C, between ≥15 °C and ≤+70 °C, and preferably between ≥20 °C and ≤+37 °C.

According to one embodiment, the method comprises further e) exchanging one or two times the pre-heated thawing medium by medium at room temperature.

According to another aspect of the invention, a recipient vessel (16) is provided comprising one or more wells or vials (9) comprising one or more frozen or vitrified biological specimen (17). At least one of the wells or vials comprises an inner volume Vᵢ that is at least 50× greater than the volume Vₛ of the biological specimen (17), including surrounding vitrified or frozen storage or culture medium, if present. According to embodiments, the inner volume Vᵢ is at least 75×, 80×, 85×, 90×, 95×, 100×, 105×, 110×, 115×, 120×, 125×, 150× or 200× greater than the volume Vₛ of the biological specimen (17).

In such way, a rapid freezing or vitrification of the specimen is assured, e.g. without harmful formation of ice crystals. It is further assured that upon dispensing the thawing medium into the recipient vessel containing the frozen biological specimen, the storage medium surrounding the biological specimen will be sufficiently diluted. This may be beneficial in order to mitigate any potentially harmful effects of ingredients of the medium on the biological specimen, like e.g. inflicted by some cryoprotective agents (of present in the medium), like DMSO.

Oftentimes, the biological specimen, e.g., a cell, a cell aggregate or cluster, or a microtissue, adopts a more or less spheroidal shape, including surrounding vitrified or frozen medium, if present. In such case, its volume can be approximated by the formula V=4/3·π·r³, in which r is the radius of the biological specimen (see FIG. 24 C).

In case a well or vial has a cylindrical shape, its volume can be approximated by the formula V=π·r²·h, in which r is the radius of the circular cross-section and h is the height of the well or vial.

In case a well or vial has a tapered or conical shape, its volume can be approximated by the formula V=(⅓)·π·h·(r₁²+r₂²+(r₁·r₂)), in which h is the height of the well or vial, r₁ is the lower radius of the vial and r₂ is the upper radius of the vial.

In case a well or vial has a semi-spherical shape, its volume can be approximated by the formula V=⅔·π·r³, in which r is the radius of the well or vial.

Regarding embodiments of the recipient vessel, the vials or wells or the biological specimen, described elsewhere herein, the features, characteristics and advantages discussed in connection with said embodiments, as well as the embodiments as such, are understood to apply to the recipient vessel and its embodiments discussed above and in the following, even if they are not repeated again.

In one embodiment, the recipient vessel is a 96 well plate, like e.g. the Akura™ 96 well plate (InSphero). In such embodiment, the wells typically have a volume of between ≥100 μl and ≤400 μl. In such embodiment, if Vᵢ is at least 100× greater than Vₛ, the biological specimen would e.g. have a maximum volume of between ≤1 μl and ≤4 μl (including surrounding vitrified or frozen storage or culture medium, if present).

In one embodiment, the recipient vessel is a 384 well plate. In such embodiment, the wells typically have a volume of between ≥80 μl and 200 μl. In such embodiment, if Vᵢ is at least 100× greater than Vₛ, the biological specimen would have a maximum volume of between ≤0.8 μl and ≤2 μl (including surrounding vitrified or frozen storage or culture medium, if present).

In one embodiment, the recipient vessel is a 1536 well plate. In such embodiment, the wells typically have a volume of between ≥10 μl and 200 μl. In such embodiment, if Vᵢ is at least 100× greater than Vₛ, the biological specimen would have a maximum volume of between ≤0.1 μl and ≤2 μl (including surrounding vitrified or frozen storage or culture medium, if present).

The following table shows some further, non-limiting examples:

| well plate | 96 | 384 |
|---|---|---|
| typical well volume range (Vi) (μl) | between ≥100 | between ≥80 |
| | and ≤400 | and ≤200 |
| Vi of Insphero Akura (μl) | 280 | 130 |
| Vs (μl) if Vi is at least 50 × greater | ≤5.6 | ≤2.6 |
| Vs (μl) if Vi is at least 80 × greater | ≤3.5 | ≤1.65 |
| Vs (μl) if Vi is at least 100 × greater | ≤2.8 | ≤1.3 |
| Vs (μl) if Vi is at least 120 × greater | ≤2.3 | ≤1.83 |
| Vs (μl) if Vi is at least 150 × greater | ≤1.87 | ≤0.87 |
| Vs (μl) if Vi is at least 200 × greater | ≤1.4 | ≤0.65 |

*Vs including surrounding vitrified or frozen storage or culture medium, if present

According to one embodiment, the recipient vessel has been produced, or is obtainable, with a method according to the above description. For such purpose, the one or more biological specimen (17) are dispensed into one or more wells or vials (9) of the recipient vessel (16) as described above.

According to one embodiment of that method, only one biological specimen (e.g., only one microtissue) is present per well or vial (9) of the recipient vessel.

### Examples

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### Materials and Methods

### 1. Methods Per Se

### 1.1. Vitrification Method

Human Tumor Microtissues (HepG2 cells), expressing eGFP, formed and cultured in an Akura™ 96 well plate (InSphero) were removed from the incubator (37 °C, 5% CO₂) and placed on the designated slot on the deck of a Hamilton Vantage liquid handling system (Hamilton AG, Bonaduz, Switzerland). The cell culturing medium was aspirated from one column of the plate and replaced with medium containing a defined mixture of cryoprotective agents (CPAs) chosen from a list of cell-permeable and cell non-permeable components (see Swain & Smith 2010, the content of which is incorporated herein by reference for enablement purposes). This process was repeated twice with increasing concentrations of CPAs. Each medium composition was left on the Microtissues for 3 min for equilibration of Microtissues and medium. After the third equilibration step, the Microtissues were picked up using an array of eight Hamilton MagPip® pipetting heads and left to sediment to the bottom of the tip.

Another Akura™ 96 well plate was pre-cooled at below −190 °C in a cryochamber according to the invention, cooled with liquid nitrogen. After equilibration and sedimentation of the Microtissues, the lid of the cryochamber was opened briefly to allow spotting of the Microtissues into the plate within a minimal medium volume in the low or sub-microliter range. The dispensing of a minimal media volume onto the pre-cooled plate led to immediate snap freezing of the medium and vitrification of the Microtissues. The cryochamber was closed again to preserve the low temperatures and the process was repeated with further columns of the same source plate.

### 1.2. Thawing Method

For thawing the plate, an array of three re-equilibration media with decreasing concentrations of CPAs (see Swain & Smith 2010) was used before exchanging the medium to standard cell culture medium. The first medium used was pre-heated to a temperature above ambient level. The frozen plate was automatically lifted out of the cryochamber and placed on a ventilated plate holder (vent plate) according to the invention, which directed pressurized air to the bottom of the plate to dissipate the cool air around the plate and its wells. The pre-heated medium was added to all 96 wells of the plate synchronously using a 96 tip pipetting head (Hamilton), leading to immediate thawing of the plate and Microtissues while minimizing damage by potential ice crystal formation while the plate, medium and Microtissues were warming up to temperatures above the freezing point.

After 1 min, the pre-heated medium was removed from the plate and replaced with the same medium at room temperature, this washing step was repeated after another minute. Subsequently, re-equilibration media 2 and 3 were added for 2 min each, with an additional refresh of the medium after the first minute of equilibration. Finally, the medium was exchanged to standard tissue culture medium and the plate placed into an incubator at 37 °C and 5% CO₂.

### 2. Experiment

Pre-formed scaffold-free microtissues are equilibrated in a defined mixture of cryoprotective agents (CPA) e.g. in an equilibration process with increasing concentrations of CPA, while a recipient vessel is pre-cooled to −196 °C in liquid nitrogen in the cryochamber. The microtissues are then spotted onto the recipient vessel (e.g. a 96-well microtissue plate; 1 microtissue per well) within small droplets (0.3-1.0 μl or 0.5-1.0 μl volume) utilizing Hamilton's MagPip pipetting technology. The small droplet containing the microtissue instantly freezes upon contact with the cold surface. This process enables cooling at the high rates required for watery samples to acquire a non-crystalline amorphic glassy state (vitrification). Vitrification renders an aqueous solution into an amorphic, glassy state preventing cell damage associated with ice crystal formation as usually observed using conventional freezing methods. The tissues can then be kept in liquid N2 gas phase at <150 °C for long-term storage.

For thawing, pre-warmed thawing medium is applied to the recipient vessel from the top. In parallel, the use of the vent plate allows warming from the bottom, enabling a fast transition from the vitrified into a liquid state, again minimizing the chance to form harmful ice crystals.

This process has been tested with pre-formed liver microtissues produced from primary human hepatocytes and non-parenchymal liver cells (see Proctor et al (2017), the content of which is incorporated herein by reference for enablement purposes). The microtissues were frozen in standard 96-well microwell plates using the cryochamber as described above. After thawing using the vent plate, the microtissues were compared to non-frozen control microtissues. The morphology showed compact and round liver microtissues similar to their non-frozen counterparts. Performing H&E staining as well as immuno-staining for, Albumin (Hepatocytes), BSEP (canalicular structures) and CD68 (Kupffer cells) revealed similar patterns as in non-frozen microtissues (see FIG. 15).

The ATP content of single microtissues was between 85% and 75% of non-frozen control tissues at day 2 and day 7 after thawing (see FIG. 16A). Thawed and control microtissues were very similar in their Albumin secretion (see FIG. 16B). CYP450 activities (1A2, 2B6, 2C9, 3A4, see FIG. 17), and responsiveness to the application of three known liver toxic compounds, as shown in a 7-day repeated dose-response toxicity study (ATP) for Chlorpromazine, Troglitazone and Tolcapone which revealed similar IC50 values for both thawed and control microtissues (FIG. 18, see also Messner et al (2013), the content of which is incorporated herein by reference for enablement purposes).

These results demonstrate the feasibility of performing cryopreservation of highly differentiated spheroids based on primary cells in standard microplates, enabling long-term storage without loss of function and that these spheroids once thawed can be directly used for drug safety testing.

### REFERENCE NUMBERS

1. first compartment
2. upper reservoir
3. second compartment
4. support for a recipient vessel
5. overflow rim
6. (second) overflow rim
7. overflow compartment
8. (third) compartment
9. one or more wells or vials
10. one or more cavities
11. positioning grid
12. lower reservoir
13. side section of upper reservoir
14. cover lid
15. opening for filling in cryogenic liquid
16. recipient vessel
17. biological specimen
18. vent plate
19. cavities in the vent plate
20. supply channel for thawing medium
21. exhaust channel
22. pipette tip
23. thawing medium
24. spacer
25. filling nozzle for cryogenic liquid
26. pusher
27. control nozzle
28. recess in overflow rim
29. flat bottom of microtiter plate
30. deflector plate
31. outer hull
32. suction port (cold air)
33. pipette array
34. input port (warm air)
35. plug to seal opening 15
36. flat air nozzle
37. tube heating device
38. air pump
39. warm air source
40. encasement
41. laminar/non turbulent flow of gas
42. hollow room formed between encasement 40 and outer hull 31
43. vitrified or frozen storage or culture medium surrounding the biological specimen 17
Vᵢ inner volume of a well or vial 9
Vₛ volume of the biological specimen 17 including surrounding vitrified or frozen storage or culture medium 43 (if present)
C cryogenic liquid
evC evaporated cryogenic liquid
A-A′ line that marks the height of the second overflow rim (6), the height of the support (4) for the recipient vessel and the height of the opening of the control nozzle (27)

### REFERENCES

Jason E. Swain and Gary D. Smith, 4-Cryoprotectants from Cryobiology, in: Ri-Cheng Chian and Patrick Quinn (eds) Fertility Cryopreservation, pp. 24-38 Cambridge University Press (2010)
Whaley D, Damyar K, Witek R P, Mendoza A, Alexander M, Lakey J R. Cryopreservation: An Overview of Principles and Cell-Specific Considerations. Cell Transplant. 2021 January-December; 30
Larsen B, 3D Cell Culture: A Review Of Current Techniques. Agilent white papers, Nov. 12, 2015. https://www.biotek.com/resources/white-papers/3d-cell-culture-a-review-of-current-techniques
Carletti E, Motta A, Migliaresi C. Scaffolds for tissue engineering and 3D cell culture. Methods Mol Biol. 2011; 695:17-39.
William R. Proctor, Alison J. Foster, Jennifer Vogt, Claire Summers, Brian Middleton, Mark A. Pilling, Daniel Shienson, Monika Kijanska, Simon Strobel, Jens M. Kelm, Paul Morgan, Simon Messner, Dominic Williams. Utility of spherical human liver microtissues for prediction of clinical drug-induced liver injury. Arch Toxicol (2017) 91:2849-2863
S Messner, I Agarkova, W Moritz, J M Kelm. Multi-cell type human liver microtissues for hepatotoxicity testing. Arch Toxicol. 2013; 87:209-13.

What is claimed is:

1. A system for freezing and/or vitrifying biological specimen (17), the system comprising
an encasement (40) arranged to accommodate a cryogenic liquid (C), and
a support (4) for a recipient vessel (16) suitable and/or arranged for accommodating one or more biological specimen (17).

2. The system according to claim 1, further comprising
a first compartment (1) and a second compartment (3)
wherein the first compartment (1) comprises an upper reservoir (2),
wherein the second compartment (3) comprises a lower reservoir (12),
wherein each reservoir (2, 12) is suitable and/or arranged to accommodate a cryogenic liquid (C),
wherein the second compartment (3) is arranged below the first compartment (1), and
wherein the second compartment comprises a support (4) for a recipient vessel (16) suitable and/or arranged for accommodating one or more biological specimen (17).

3. The system according to any one of the aforementioned claims, wherein the system further comprises a recipient vessel (16) suitable and/or arranged for accommodating one or more biological specimen (17).

4. The system according to any one of the aforementioned claims, wherein the upper reservoir (2) comprises a first overflow rim (5) arranged to allow the cryogenic liquid (C) to flow into the lower reservoir (12) if the level height of the cryogenic liquid (C) in the upper reservoir (2) exceeds the height of the first overflow rim (5).

5. The system according to any one of the aforementioned claims, wherein the second compartment (3) comprises a second overflow rim (6) arranged to create, within the second compartment (3), an overflow compartment (7), wherein the second overflow rim (6) is arranged to allow the cryogenic liquid (C) to flow from the lower reservoir (12) within the second compartment into the overflow compartment (7) if the level height of the cryogenic liquid (C) in the lower reservoir (12) exceeds the height of the second overflow rim (6).

6. The system according to any one of the aforementioned claims, wherein the height of the second overflow rim (6) aligns with the height of the support (4) for the recipient vessel (16).

7. The system according to any one of the aforementioned claims, wherein the system further comprises a third compartment (8) arranged below the second compartment (3), which third compartment (8) is in fluid communication with the second compartment (3) and/or the overflow compartment (7), and is suitable and/or arranged to accommodate a cryogenic liquid (C).

8. The system according to any one of the aforementioned claims, wherein the system further comprises a cover lid (14) comprising a first opening (15) suitable and/or arranged for filling in a cryogenic liquid (C) into the upper reservoir (2) of the first compartment (1).

9. The system according to any one of the aforementioned claims, wherein the system further comprises a filling nozzle (25) for the cryogenic liquid (C) which is suitable and/or arranged for filling the cryogenic liquid (C) directly into the second compartment (3) and/or the overflow compartment (7) and/or into the third compartment (8).

10. The system according to any one of the aforementioned claims, wherein the system further comprises a control nozzle (27) with an opening, wherein the height of the opening of the control nozzle aligns with the height of the second overflow rim (6) of the second compartment (3).

11. The system according to any one of the aforementioned claims, wherein the support (4) for the recipient vessel (16) comprises one or more cavities (10) on its upper side that are suitable and/or arranged to accommodate the one or more wells or vials (9) of the recipient vessel (16) suitable and/or arranged for accommodating biological specimen.

12. The system according to any one of the aforementioned claims, wherein the one or more cavities (10) are arranged to be essentially form-locking with at least part of the one or more wells or vials (9) of the recipient vessel (16).

13. The system according to any one of the aforementioned claims, wherein the support (4) for the recipient vessel comprises a material that has a wicking effect on the cryogenic liquid.

14. The system according to any one of the aforementioned claims, wherein the support (4) for the recipient vessel comprises a material that has an open-cell sponge-like structure.

15. The system according to any one of the aforementioned claims, wherein the support (4) for the recipient vessel comprises a material that maintains elastic deformability at a temperature below −70 °C and/or in the presence of the cryogenic liquid (C).

16. The system according to any one of the aforementioned claims, wherein the support (4) for the recipient vessel has an absolute height of between ≥5 and ≤50 mm.

17. The system according to any one of the aforementioned claims, wherein the support (4) for the recipient vessel comprises a material selected from the group consisting of
melamine (1,3,5-Triazine-2,4,6-triamine) or melamine resin,
polyurethane, and/or
cross linked polyolefin foam

18. The system according to any one of the aforementioned claims, wherein the third compartment (8) comprises a material that has at least one of the following properties:
a) a wicking effect on the cryogenic liquid,
b) an open-cell sponge-like structure,
c) maintains elastic deformability at a temperature below −70 °C and/or in the presence of the cryogenic liquid, and/or
d) comprises a material selected from the group consisting of
melamine (1,3,5-Triazine-2,4,6-triamine) or melamine resin,
polyurethane, and/or
cross linked polyolefin foam

19. The system according to any one of the aforementioned claims, wherein the cryogenic liquid (C) is selected from the group consisting of
liquid nitrogen,
liquid helium,
liquid argon,
liquid oxygen,
a mixture of dry ice and an organic solvent.

20. The system according to any one of the aforementioned claims, wherein the support (4) is a support according to any one of claims 13-18.

21. The system according to any one of the aforementioned claims, which system further comprises pipetting means (22, 33) for dispensing biological specimen into one or more wells or vials (9) of the recipient vessel (16).

22. The system according to claim 23, which system further comprises ventilation means (30, 32, 34, 36, 37, 38, 39) to generate a warm gas current along the pipette tips (22) of the pipetting means (33).

23. The system according to claim 24, wherein such ventilation means comprise at least one element selected from the group consisting of
deflector plate (30)
air suction port (32)
air input port (34)
flat air nozzle (36)
tube heating device (37)
air pump (38)
warm water bath (39).

24. The system according to any one of the aforementioned claims, wherein the support (4) for the recipient vessel is arranged on one or more spacers (24).

25. The system according to any one of the aforementioned claims, wherein the recipient vessel (16) comprises one or more wells or vials (9) suitable and/or arranged for accommodating biological specimen.

26. The system according to any one of the aforementioned claims, wherein the recipient vessel (16) is a microtiter plate or an array of vials corresponding to a 6, 24, 96, or 384 microtiter plate layout.

27. A support (4) for freezing or vitrifying of biological specimen comprised in a recipient vessel (16), which support comprises a material that has a wicking effect on a cryogenic liquid.

28. The support according to claim 27, wherein the cryogenic liquid (C) is selected from the group consisting of
liquid nitrogen,
liquid helium,
liquid argon,
liquid oxygen,
a mixture of dry ice (CO₂) and an organic solvent.

29. The support according to any one of the aforementioned claims, which comprises a material that has an open-cell sponge-like structure.

30. The support according to any one of the aforementioned claims, which comprises a material that maintains elastic deformability at a temperature below −70 °C and/or in the presence of the cryogenic liquid.

31. The support according to any one of the aforementioned claims, which has an absolute height of between ≥5 and ≤50 mm.

32. The support according to any one of the aforementioned claims, which comprises a material selected from the group consisting of
melamine (1,3,5-Triazine-2,4,6-triamine) or melamine resin,
polyurethane, and/or
cross linked polyolefin foam

33. The support according to any one of the aforementioned claims, which comprises one or more cavities (10) on its upper side that are suitable and/or arranged to accommodate one or more wells or vials (9) of a recipient vessel (16) suitable and/or arranged for accommodating biological specimen.

34. The support according to claim 33, wherein the one or more cavities (10) are arranged to be essentially form-locking with at least part of the one or more wells or vials (9).

35. The support according to any one of the aforementioned claims, wherein the recipient vessel (16) is a microtiter plate or an array of vials corresponding to a 6, 24, 96, or 384 microtiter plate layout.

36. A vent plate (18) for thawing of biological specimen (17) comprised in a recipient vessel (16), which plate comprises one or more cavities (19) on its upper side that are suitable and/or arranged to accommodate one or more wells or vials (9) of a recipient vessel (16) suitable and/or arranged for accommodating biological specimen (17), the vent plate (18) further comprising
one or more supply channels (20) suitable and/or arranged to supply a thawing medium to the outer walls of the wells or vials (9) of the recipient vessel (16), and
one or more exhaust channels (21) suitable and/or arranged to drain off thawing medium from the outer walls of the wells or vials (9) of the recipient vessel.

37. The vent plate (18) according to claim 36, wherein the thawing medium (23) is a fluid, liquid or gas.

38. The vent plate according to any one of claims 36-37, wherein the thawing medium (23) comprises a temperature of between ≥−150 °C and ≤+100 °C.

39. A system for thawing of biological specimen comprised in a recipient vessel (16), which system comprises
a) a vent plate (18) according to any one of claims 36-38, and
b) pipetting means (22) suitable and/or arranged for dispensing, into the wells or vials (9) of the recipient vessel (16), a thawing medium (23).

40. The system, vent plate (18) or support (4) according to any of the aforementioned claims, wherein the biological specimen (17) is at least one selected from the group consisting of
one or more tissue samples,
one or more gametocytes (oocytes or spermatocyte),
one or more blastocysts,
one or more individual cells,
one or more cellular aggregates (e.g. microtissues, spheroid), and/or
one or more organoids derived from stem cells by self-organization and differentiation.

41. A method for freezing and/or vitrifying biological specimen (17), said method comprising the steps of
a) placing a recipient vessel (16) suitable and/or arranged for accommodating one or more biological specimen (17) on a support (4) according to any one of claims 13-18, the support (4) being arranged in a system according to any one of claims 27-35
b) before or after step a), dispensing one or more biological specimen (17) into one or more wells or vials (9) of the recipient vessel (16), and
c) before or after step a) or b), filling a cryogenic liquid (C) into the system according to any one of claims 1-26

42. The method according to claim 34, which further comprises the step of adding one or more cryoprotective agents to the biological specimen before step b).

43. A method of storing biological specimen frozen and/or vitrified with a method according to claim 34 or 35, the method comprising the steps of
a) keeping the recipient vessel (16) which accommodates one or more frozen and/or vitrified biological specimen (17) within the system according to any one of claims 1-26,
b) optionally, positioning a lid (14) on top of the system according to any one of claims 1-26, and
c) optionally, replenishing cryogenic liquid (C) into the system according to any one of claims 1-26

44. A method of thawing biological specimen (17) frozen and/or vitrified with a method according to claim 39 or 40 and/or stored with the method according to claim 43, the method comprising the steps of:
a) removing the recipient vessel (16) from the support (4)
b) placing the recipient vessel (16) on a vent plate (18) according to any one of claims 36-38
c) supplying the vent plate (18) with a thawing medium (23)
optionally, simultaneously with step c) or sequentially prior or after step c), dispensing a thawing medium (23) from pipetting means (22, 33) arranged above of the recipient vessel (16).

45. A recipient vessel (16) comprising one or more wells or vials (9) comprising one or more frozen or vitrified biological specimen (17), wherein at least one of the wells or vials (9) comprises an inner volume Vᵢ that is at least 100× greater than the volume Vₛ of the biological specimen (17), including surrounding vitrified or frozen storage or culture medium, if present.

46. The recipient vessel according to claim 45, which has been produced, or is obtainable, with a method according to any one of claims 41-42
